(19) United States
(12) Patent Application Publication
(10) Pub. No.: US 2022/0002458 A1
(43) Pub. Date: Jan. 6, 2022

(54) ACRYLATE COPOLYMERS FOR GALENIC APPLICATIONS

(71) Applicant: JOHANNES GUTENBERG-UNIVERSITÄT MAINZ, Mainz (DE)

(72) Inventors: Holger FREY, Emmendingen (DE); Erik KERSTEN, Mainz (DE); Peter LANGGUTH, Biebergemünd (DE); Johannes Andreas BLECHAR, Frankfurt a.M. (DE); Jozef AL-GOUSOUS, Mainz (DE)

(73) Assignee: JOHANNES GUTENBERG-UNIVERSITÄT MAINZ, Mainz (DE)

(21) Appl. No.: 17/294,049

(22) PCT Filed: Oct. 19, 2019
(86) PCT No.: PCT/EP2019/077501

§ 371 Date: May 14, 2021

(30) Foreign Application Priority Data
Nov. 22, 2018 (DE) 10 2018 129 419.0

(51) Int. Cl. C08F 220/14; C08F 8/12; A61K 9/28; A61K 9/48; C08F 220/18; C08F 4/80; C08F 220/28; C08F 2/18; C08F 220/06

(52) U.S. Cl. CPC C08F 220/14; C08F 8/12; A61K 9/284; A61K 9/4891; C08F 220/06; C08F 4/80; C08F 220/283; C08F 2/18; C08F 220/18

(57) ABSTRACT

The invention relates to a copolymer for galenic applications, having an acrylate backbone and sidearms, containing α-hydroxycarboxylic acid groups. The invention further relates to the process of preparing the foregoing copolymer for galenic applications and its use thereof.

The present invention relates to a process for preparing acrylate copolymers, to acrylate copolymers produced by the process, and to the use thereof for pharmaceutical formulations and coatings.

The prior art includes known acrylate polymers for pharmaceutical applications. For example, Evonik Industries AG and BASF SE supply acrylate copolymers for tablet coatings under the product names Eudragit® and Kollicoat®. Table 1 contains an overview of various Eudragit® copolymers.

TABLE 1
Eudragit ® copolymers

| Eudragit ® | Composition | CAS No. |
|---|---|---|
| L 100 | Poly(methacrylic acid-co-methyl | 25086-15-1 |
| | methacrylate) 1:1 | |
| S 100 | Poly(methacrylic acid-co-methyl | 25086-15-1 |
| | methacrylate) 1:2 | |
| L 100-55 | Poly(methacrylic acid-co-ethyl | 25212-88-8 |
| | acrylate) 1:1 | |
| L 30 D-55 | Poly(methacrylic acid-co-ethyl | 25212-88-8 |
| | acrylate) 1:1 | |
| L 12.5 | Poly(methacrylic acid-co-methyl | 25086-15-1 |
| | methacrylate) 1:1 | |
| S 12.5 | Poly(methacrylic acid-co-methyl | 25086-15-1 |
| | methacrylate) 1:2 | |
| FS 30 D | Poly(methyl acrylate-co-methyl | 26936-24-3 |
| | methacrylate-co-methacrylic acid) | |
| | 7:3:1 | |
| NE 30 D | Poly(ethyl acrylate-co-methyl | 9010-88-2 |
| | methacrylate) 2:1 | |

DE 10 2005 010 108 A1 relates to water-soluble polymers for cosmetic or pharmaceutical applications, and discloses copolymers having monomer units (m1) and (m2), where (m1) is selected from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, and (m2) has the structure

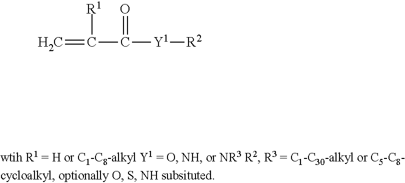

EP 2 679 216 B1 discloses a core-shell tablet for multiphase release of betahistine, having an interlayer, disposed between core and shell, with a film former composed of cellulose derivatives, methacrylic acid polymers, polyvinyl derivatives and mixtures thereof, wherein core and/or shell preferably comprise(s) an α-hydroxycarboxylic acid as buffer.

WO 2015/000970 A1 relates to a process and to a polymer synthesized by the process and to the use thereof for pharmaceutical formulations. In the process, by means of free-radical polymerization, an α,β-ethylenically unsaturated carboxylic acid, sulfonic acid and/or phosphonic acid and a crosslinking monomer are copolymerized with a polyether component. Among the copolymers obtained are those composed of polyacrylic acid and polyethers.

Moreover, DE69510190T2 describes a bioadhesive (or mucoadhesive) pharmaceutical composition in the form of a spray comprising a fatty acid ester with a saturated or unsaturated fatty acid having a total number of carbon atoms of 8 to 22, where the fatty acid ester is selected from fatty acid esters of polyhydric alcohols, hydroxycarboxylic acids, monosaccharides, glyceryl phosphate derivatives, glyceryl sulfate derivatives and mixtures of the aforementioned fatty acid esters.

The tablet is the most commonly used drug form with a market share of nearly fifty percent. Reasons for this are the simple and inexpensive production and packaging, precise active ingredient dosage, long shelf life, and ease of storage, handling and taking for patients, which are associated with good therapeutic compliance. Moreover, numerous active ingredients are suitable for pressing in tablet form with pharmaceutical auxiliaries.

A prerequisite for peroral administration of medicaments and absorption in the digestive tract is a certain hydrophilicity of the active ingredient, associated with water solubility. A tablet coating is used for protection of the ingredients of a tablet from moisture and outside influences, and for flavor masking. Numerous active pharmaceutical ingredients are alkaloids, and have an unpleasantly bitter taste.

In the case of peroral administration, it is sometimes an absolute necessity to protect the active pharmaceutical ingredient from the harsh conditions of the stomach. The pH of the empty stomach is about 2, and in the event of food intake can rise to values above 4.5. In the case of acid-labile active ingredients, for example omeprazole, this can lead to irreversible changes. For a number of medicaments (e.g. 5-aminosalicylic acid), the therapeutic aim is controlled release in a defined region of the digestive tract. Specialists frequently also refer to controlled release as “drug targeting”. Moreover, there are active ingredients that irritate the gastric mucosa (e.g. acetylsalicylic acid), for which a gastric juice-resistant coating is indicated in order to reduce gastric side effects. The use of gastric juice-resistant coatings is not limited to tablets. Other oral drug formulations, such as capsules and granules, are also coated with gastric juice-resistant coatings. For gastric juice-resistant coatings, preference is given to using slightly acidic copolymers that are in protonated form and hence sparingly soluble in the stomach. The market for pharmaceutical formulations and coatings was long dominated by cellulose acetate phthalates (CAP), which in recent times have increasingly been displaced by methacrylic acid-ethyl acrylate copolymers.

Eudragit® polymers (Evonik Industries AG) are among a group of acrylate copolymers that were developed in the 1950s for use as tablet coating and carrier material for tablets. All Eudragit® polymers have the common feature of a polyacrylate or polymethacrylate backbone. Depending on the type, the Eudragit® polymers differ in the substitution pattern of the side chain and in their dissolution characteristics. Eudragit® analog polymers are sold by BASF SE under the Kollicoat® product name. Tablet coatings based on acrylate copolymers feature mechanical stability, a high water vapor barrier and acid stability.

According to manufacturer data, the solubility of the established acrylate copolymers increases rapidly over and above a pH of 5.5. However, in vivo studies show that the solvation of tablet coatings based on acrylate copolymers and the associated active ingredient release are too slow for targeting of the duodenum (Cole, E. T.; Scott, R. A.; Connor, A. L.; Wilding, I. R.; Petereit, H.-U.; Schminke, C.; Beckert, T.; Cadé, D. International Journal of Pharmaceutics 2002, 231 (1), 83-95. DOI: 10.1016/50378-5173(01)00871-7; Al-Gousous, J.; Amidon, G. L.; Langguth, P. Molecular pharmaceutics 2016, 13 (6), 1927-1936; DOI: 10.1021/acs.molpharmaceut.6b00077; Liu, F.; Basit, A. W. Journal of controlled release: official journal of the Controlled Release Society 2010, 147 (2), 242-245; DOI: 10.1016/j.jconre1.2010.07.105). This is particularly problematic for active ingredients that are absorbed primarily in the duodenum.

Further therapeutic problems are caused by non-site-specific active ingredient release. In this connection, mention may be made by way of example of the enzyme pancreatin, which is administered to patients having exocrine pancreatic insufficiency. If acid-labile pancreatin is not released immediately downstream of the stomach, there is an increased incidence of intestinal complaints because lipids present in food are not fully digested.

In view of the problems described above, there is a need for pharmaceutical coatings that dissolve more rapidly after departure from the stomach than the materials known in the prior art.

In the context of the inventive process, novel acrylate monomers of the Ayl—O—R—OP or MAyl—O—R—OP type are synthesized, in which “Ayl” is acryloyl, “MAyl” is methacryloyl, “R” is a residue of a α-hydroxycarboxylic acid and “P” is a protecting group. The α-hydroxycarboxylic acid is selected from hydroxyethanoic acid (glycolic acid), 2-hydroxypropanoic acid (lactic acid), 2-hydroxybutanoic acid, 2-hydroxyisobutanoic acid, 2-hydroxy-2-methyl-3-oxobutanoic acid, phenylhydroxyethanoic acid, 2-hydroxy-4-methylthiobutanoic acid, 2-hydroxybutane-1,4-dioic acid (malic acid), 2-hydroxypropanedioic acid, 2-hydroxypropane-1,2,3-tricarboxylic acid, hydroxypropane-1,2,3-tricarboxylic acid or 2,3-dihydroxybutanedioic acid and has the structure OH—R—OH in which R is —CH₂(C═O)—, —CH(CH₃)(C═O)—, —CH(CH₂CH₃)(C═O)—, —C(CH₃)₂(C═O)—, —C(CH₃)(COCH₃)(C═O)—, —CH(Ph)(C═O)—, —CH[(CH₂)₂SCH₃](C═O)—, —CH(CH₂COOH)(C═O)—, —CH(COOH)(C═O)—, —C(CH₂COOH)₂(C═O)—, —CH(COOH)CH(CH₂COOH)(C═O)— or —CH(COOH)(CHOH)(C═O)—. The protecting group P is a benzyl group (—CH₂Ph), a tert-butyl group (—C(CH₃)₃) or an allyl group. In expedient embodiments of the process of the invention, the protected monomers synthesized are methacryloyloxyethanoate benzyl (MAylO-Gly-Bn), (2S)-2-methacryloyloxypropionate benzyl (MAylO-L-La-Bn) and (R,S)-2-methacryloyloxypropionate benzyl (MAylO-D,L-La-Bn). The protected monomers referred to above are copolymerized by means of free-radical polymerization with methyl acrylate (MA), methyl methacrylate (MMA), ethyl acrylate (EA) or ethyl methacrylate (EMA), and optionally with protected acrylic acid or protected methacrylic acid. Subsequently, the protecting group P is removed by reduction from the synthesized copolymers. For example, in the case that P=Bn, the benzyl protecting group is substituted by hydrogenolysis by means of heterogeneous palladium catalysis in a hydrogen atmosphere.

The esterification of the α-hydroxycarboxylic acid with acrylic acid or methacrylic acid or with the functional sidearms of a polymer from the Eudragit® family is conducted in three steps: (i) introducing a protecting group, (ii) esterifying and (iii) deprotecting, in order to avoid the formation of oligomers of the bifunctional α-hydroxycarboxylic acid. First of all, in step (i), the carboxyl function of the α-hydroxycarboxylic acid is blocked by means of a protecting group, for example benzyl (Bn). The benzyl protecting group is acid- and base-stable and is removed by hydrolysis in step (iii) after the esterification of the protected α-hydroxycarboxylic acid in step (ii).

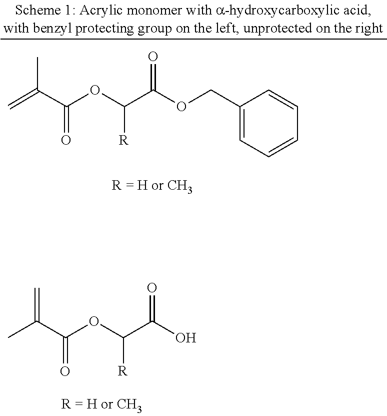

In order to obtain the benefits of established acrylate copolymers for use as gastric juice-resistant tablet coating, the base structure of the approved Eudragit® polymers is built on. The poly(meth)acrylate backbone is retained and the side chain is modified.

It has been found that, completely surprisingly, even a slight modification to the established acrylate copolymers in which a proportion of only 5 to 20% of the carboxyl OH groups is replaced by residues of an α-hydroxycarboxylic acid, especially by glycolic acid or lactic acid residues, significantly influences the dissolution characteristics and moves them in the direction of low pH values. It has not been possible to date to satisfactorily clarify the abrupt change in the mechanism of action in the replacement of carboxyl groups by α-hydroxy-carboxylic acid residues. It is suspected that at least a portion of the replacement sites, on account of steric effects, increase the free volume, and there is an increase in the relaxation (mobility) of polymer chains. The substituents act like an internal plasticizer.

The starting point is the hypothesis that a negative inductive effect acts on the carboxyl group of the α-hydroxycarboxylic acid substituents. The resonance structure shown in scheme 2 illustrates the induction brought about by the partial positive charge of the oxygen atom in the carboxyl group.

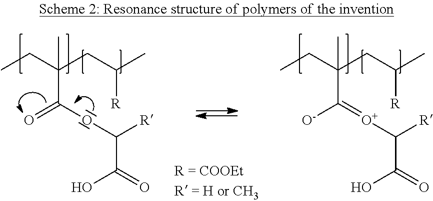

The invention encompasses the synthesis of copolymers having monomer units containing residues of an α-hydroxycarboxylic acid, for example glycolic acid, L-lactic acid or D,L-lactic acid. In an expedient embodiment, the copolymers of the invention have a structure that embodies an analog modification of the acrylate copolymers of the Eudragit® or Kollicoat® type.

A considerable advantage of the polymers of the invention is good physiological compatibility. Hydrolytic cleavage of the ester bond releases glycolic acid or lactic acid in the gastrointestinal tract. Lactic acid is an endogenous substance and is approved as food additive (E 270). Glycolic acid has very low, physiologically irrelevant toxicity. Studies show that the polymers of the invention have a higher solubility than Eudragit® polymers at pH values of 4 to 5.

For qualitative determination of the solubility, the polymers of the invention are suspended at room temperature in a snap-lid bottle with buffer solution in a concentration of 5 mg of polymer per mL of buffer solution. Solvation proceeds either within a few minutes (table 2: +sign) or is virtually completely absent—even in the case of suspension in the buffer solution for several days (table 2: −sign).

TABLE 2
Solubility

| Polymer | pH 2 | pH 3 | pH 4 | pH 5 | pH 6 | pH 7 |
|---|---|---|---|---|---|---|
| Eudragit ® L 100 | − | − | − | − | + | + |
| Eudragit ® L 100-55 | − | − | − | − | + | + |
| poly(MAylO-Gly-co-EA) | − | − | − | + | + | + |
| poly(MAylO-L-La-co-EA) | − | − | − | + | + | + |

Eudragit® L 100 (Evonik Industries AG) and an analog polymer prepared by means of controlled free-radical polymerization (CFRP), referred to as “L 100 analog” or “MA-co-EA”, have virtually the same dissolution characteristics.

By contrast, Eudragit® polymers that have been modified with an α-hydroxycarboxylic acid, such as glycolic acid, L-lactic acid or D,L-lactic acid, of the “MAylO-Gly-co-EA”, “MAylO-L-La-co-EA” and “MAylO-D,L-La-co-EA” type, dissolve at lower pH than the known Eudragit® polymers and thus provide the basis for gastric juice-resistant formulations having faster active ingredient release and absorption.

As set out above, it is an object of the invention to provide polymers for pharmaceutical formulations that have different dissolution characteristics than known acrylate copolymers. This object is achieved by a copolymer having the following structure:
poly(MAₓ-co-[AS—(O—R)ₙ—OH]_{y}-co-[AS—OH]_{z}),
poly(MMAₓ-co-[AS—(O—R)ₙ—OH]_{y}-co-[AS—OH]_{z}),
poly(EAₓ-co-[AS—(O—R)ₙ—OH]_{y}-co-[AS—OH]_{z}),
poly(EMAₓ-co-[AS—(O—R)ₙ—OH]_{y}-co-[AS—OH]_{z}),
poly(MAₓ-co-[AS—(O—R)ₙ—OH]_{y}-co-[MAS—OH]_{z}),
poly(MMAₓ-co-[AS—(O—R)ₙ—OH]_{y}-co-[MAS—OH]_{z}),
poly(EAₓ-co-[AS—(O—R)ₙ—OH]_{y}-co-[MAS—OH]_{z}),
poly(EMAₓ-co-[AS—(O—R)ₙ—OH]_{y}-co-[MAS—OH]_{z}),
poly(MAₓ-co-[MAS—(O—R)ₙ—OH]_{y}-co-[AS—OH]_{z}),
poly(MMAₓ-co-[MAS—(O—R)ₙ—OH]_{y}-co-[AS—OH]_{z}),
poly(EAₓ-co-[MAS—(O—R)ₙ—OH]_{y}-co-[AS—OH]_{z}),
poly(EMAₓ-co-[MAS—(O—R)ₙ—OH]_{y}-co-[AS—OH]_{z}),
poly(MAₓ-co-[MAS—(O—R)ₙ—OH]_{y}-co-[MAS—OH]_{z}),
poly(MMAₓ-co-[MAS—(O—R)ₙ—OH]_{y}-co-[MAS—OH]_{z}),
poly(EAₓ-co-[MAS—(O—R)ₙ—OH]_{y}-co-[MAS—OH]_{z}),

or
poly(EMAₓ-co-[MAS—(O—R)ₙ—OH]_{y}-co-[MAS—OH]_{z}),

in which MA=methyl acrylate residue (—CH[(C═O)OCH₃]CH₂—), MMA=methyl methacrylate residue (—C(CH₃)[(C═O)OCH₃]CH₂—), EA=ethyl acrylate residue (—CH[(C═O)OCH₂CH₃]CH₂—), EMA=ethyl methacrylate residue (—C(CH₃)[(C═O)OCH₂CH₃]CH₂—), AS=acrylic acid residue (—CH[(C═O)—]CH₂—), MAS=methacrylic acid residue (—C(CH₃)[(C═O)—]CH₂—);
R=—CH₂(C═O)—, R=—CH(CH₃)(C═O)—, R=—CH(CH₂CH₃)(C═O)—, R=—C(CH₃)₂(C═O)—, R=—C(CH₃(COCH₃)(C═O)—, R=—CH(Ph)(C═O)—, R=—[(CH₂)₂SCH₃](C═O)—, R=—CH(CH₂COOH)(C═O)—, R=—CH(COOH)(C═O)—, R=—C(CH₂COOH)₂(C═O)—, R=—CH(COOH)CH(CH₂COOH)(C═O)— or R=—CH(COOH)(CHOH)(C═O)—;
n is an integer with 1≤n≤20 and x, y, z denote the relative molar proportions of the monomer units with 1≤x≤20, 1≤y23 20 and 0≤z≤20.

In expedient embodiments of the invention,
the copolymer has the structure
   poly(MAₓ-stat-[AS—(O—R)ₙ—OH]_{y}-stat-[AS—OH]_{z}),
   poly(MMAₓ-stat-[AS—(O—R)ₙ—OH]_{y}-stat-[AS—OH]_{z}),
   poly(EAₓ-stat-[AS—(O—R)ₙ—OH]_{y}-stat-[AS—OH]_{z}),
   poly(EMAₓ-stat-[AS—(O—R)ₙ—OH]_{y}-stat-[AS—OH]_{z}),
   poly(MAₓ-stat-[AS—(O—R)ₙ—OH]_{y}-stat-[MAS—OH]_{z}),
   poly(MMAₓ-stat-[AS—(O—R)ₙ—OH]_{y}-stat-[MAS—OH]_{z}),
   poly(EAₓ-stat-[AS—(O—R)ₙ—OH]_{y}-stat-[MAS—OH]_{z}),
   poly(EMAₓ-stat-[AS—(O—R)ₙ—OH]_{y}-stat-[MAS—OH]_{z}),
   poly(MAₓ-stat-[MAS—(O—R)ₙ—OH]_{y}-stat-[AS—OH]_{z}),
   poly(MMAₓ-stat-[MAS—(O—R)ₙ—OH]_{y}-stat-[AS—OH]_{z}),
   poly(EAₓ-stat-[MAS—(O—R)ₙ—OH]_{y}-stat-[AS—OH]_{z}),
   poly(EMAₓ-stat-[MAS—(O—R)ₙ—OH]_{y}-stat-[AS—OH]_{z}),
   poly(MAₓ-stat-[MAS—(O—R)ₙ—OH]_{y}-stat-[MAS—OH]_{z}),
   poly(MMAₓ-stat-[MAS—(O—R)ₙ—OH]_{y}-stat-[MAS—OH]_{z}),
   poly(EAₓ-stat-[MAS—(O—R)ₙ—OH]_{y}-stat-[MAS—OH]_{z}) or
   poly(EMAₓ-stat-[MAS—(O—R)ₙ—OH]_{y}-stat-[MAS—OH]_{z});
n=1, n=2, n=3, n=4, n=5, n=6, n=7, n=8, n=9, n=10, n=11, n=12, n=13, n=14, n=15, n=16, n=17, n=18, n=19 or n=20;
n=1, n=2 or n=3;
x is a real number;
1≤x≤12 or 8≤x≤20;
1≤x≤6,4≤x≤8, 6≤x≤10, 8≤x≤12, 10≤x≤14, 12≤x≤16, 14≤x≤18 or 16≤x≤20;
1≤x≤3, 2≤x≤4, 3≤x≤5, 4≤x≤6, 5≤x≤7, 6≤x≤8, 7≤x≤9, 8≤x≤10, 9≤x≤11, 10≤x≤12, 11≤x≤13, 12≤x≤14, 13≤x≤15, 14≤x≤16, 15≤x≤17, 16≤x≤18, 17≤x≤19 or 18≤x≤20;
x=1, x=2, x=3, x=4, x=5, x=6, x=7, x=8, x=9, x=10, x=11, x=12, x=13, x=14, x=15, x=16, x=17, x=18, x=19, x=20;
y is a real number;
1≤y≤12 or 8≤y≤20;
1≤y≤6, 4≤y≤8, 6≤y≤10, 8≤y≤12, 10≤y≤14, 12≤y≤16, 14≤y≤18 or 16≤y≤20;
1≤y≤3, 2≤y≤4, 3≤y≤5, 4≤y≤6, 5≤y≤7, 6≤y≤8, 7≤y≤9, 8≤y≤10, 9≤y≤11, 10≤y≤12, 11≤y≤13, 12≤y≤14, 13≤y≤15, 14≤y≤16, 15≤y≤17, 16≤y≤18, 17≤y≤19 or 18≤y≤20;
y=1, y=2, y=3, y=4, y=5, y=6, y=7, y=8, y=9, y=10, y=11, y=12, y=13, y=14, y=15, y=16, y=17, y=18, y=19 or y=20;
z is a real number;
z=0;
1≤z≤12 or 8≤z≤20;
1≤z≤6, 4≤z≤8, 6≤z≤10, 8≤z≤12, 10≤z≤14, 12≤z≤16, 14≤z≤18 or 16≤z≤20;
1≤z≤3, 2≤z≤4, 3≤z≤5, 4≤z≤6, 5≤z≤7, 6≤z≤8, 7≤z≤9, 8≤z≤10, 9≤z≤11, 10≤z≤12, 11≤z≤13, 12≤z≤14, 13≤z≤15, 14≤z≤16, 15≤z≤17, 16≤z≤18, 17≤z≤19 or 18≤z≤20;
z=1, z=2, z=3, z=4, z=5, z=6, z=7, z=8, z=9, z=10, z=11, z=12, z=13, z=14, z=15, z=16, z=17, z=18, z=19 or z=20;
0.8·x≤y+z≤1.2·x;
0.9·x≤y+z≤1.1·x;
y+z=x;
0.3·x≤y+z≤0.7·x;
0.4·x≤y+z≤0.6·x;
y+z=0.5·x;
y≤z≤6·y, 5·y≤z≤11·y, 8·y≤z≤14·y, 11·y≤z≤17·y or 14·y≤z≤20·y;
y≤z≤3·y, 2·y≤z≤4·y, 3·y≤z≤5·y, 4·y≤z≤6·y, 5·y≤z≤7·y, 6·y≤z≤8·y, 7·y≤z≤9·y, 8·y≤z≤10·y, 9·y≤z≤11·y, 10·y≤z≤12·y, 11·y≤z≤13·y, 12·y≤z≤14·y, 13·y≤z≤15·y, 14·y≤z≤16·y, 15·y≤z≤17·y, 16·y≤z≤18·y, 17·y≤z≤19·y or 18·y≤z≤20·y;
the copolymer has the structure poly(MAₓ-co-[AS—O—R—OH]_{y}-co-[AS—OH]_{z});
the copolymer has the structure poly(MAₓ-co-[MAS—O—R—OH]_{y}-co-[MAS—OH]_{z});
the copolymer has the structure poly(MMAₓ-co-[AS—O—R—OH]_{y}-co-[AS—OH]_{z});
the copolymer has the structure poly(MMAₓ-co-[MAS—O—R—OH]_{y}-co-[MAS—OH]_{z});
the copolymer has the structure poly(EAₓ-co-[AS—O—R—OH]_{y}-co-[AS—OH]_{z});
the copolymer has the structure poly(EAₓ-co-[MAS—O—R—OH]_{y}-co-[MAS—OH]_{z});
the copolymer has the structure poly(EMAₓ-co-[AS—O—R—OH]_{y}-co-[AS—OH]_{z});
the copolymer has the structure poly(EMAₓ-co-[AS—O—R—OH]_{y}-co-[MAS—OH]_{z});
the copolymer has the structure poly(MA₁-co-[MAS—O—R—OH]₁);
the copolymer has the structure poly(MA₂-co-[MAS—O—R—OH]₁);
the copolymer has the structure poly(MA₁-co-[MAS—O—R—OH]₂);
the copolymer has the structure poly(MMA₁-co-[MAS—O—R—OH]₁);
the copolymer has the structure poly(MMA₂-co-[MAS—O—R—OH]₁);
the copolymer has the structure poly(MMA₁-co-[MAS—O—R—OH]₂);
R═—CH₂(C═O)—;
R═—CH(CH₃)(C═O)—;
the copolymer has a molar mass M_{w} with 4000 g·mol⁻¹≤M_{w}≤500 000 g·mol⁻¹;
the copolymer has a molar mass M_{w} with 4000 g·mol⁻¹≤M_{w}≤30 000 g·mol⁻¹, 20 000 g·mol⁻¹≤M_{w}≤60 000 g·mol⁻¹, 40 000 g·mol⁻¹≤M_{w}≤80 000 g·mol⁻¹, 60 000 g·mol⁻¹≤M_{w}≤100 000 g·mol⁻¹, 80 000 g·mol⁻¹≤M_{w}≤120 000 g·mol⁻¹, 100 000 g·mol⁻¹≤M_{w}≤140 000 g·mol⁻¹, 120 000 g·mol⁻¹≤M_{w}≤160 000 g·mol⁻¹, 140 000 g·mol⁻¹≤M_{w}≤180 000 g·mol⁻¹, 160 000 g·mol⁻¹≤M_{w}≤200 000 g·mol⁻¹, 180 000 g·mol⁻¹≤M_{w}≤220 000 g·mol⁻¹, 200 000 g·mol⁻¹≤M_{w}≤240 000 g·mol⁻¹, 220 000 g·mol⁻¹≤M_{w}≤260 000 g·mol⁻¹, 240 000 g·mol⁻¹≤M_{w}≤280 000 g·mol⁻¹, 260 000 g·mol⁻¹≤M_{w}≤300 000 g·mol⁻¹, 280 000 g·mol⁻¹≤M_{w}≤320 000 g·mol⁻¹, 300 000 g·mol⁻¹≤M_{w}≤340 000 g·mol⁻¹, 320 000 g·mol⁻¹≤M_{w}≤360 000 g·mol⁻¹, 340 000 g·mol⁻¹≤M_{w}≤380 000 g·mol⁻¹, 360 000 g·mol⁻¹≤M_{w}≤400 000 g·mol⁻¹, 380 000 g·mol⁻¹≤M_{w}≤420 000 g·mol⁻¹, 400 000 g·mol⁻¹≤M_{w}≤440 000 g·mol⁻¹, 420 000 g·mol⁻¹≤M_{w}≤460 000 g·mol⁻¹, 440 000 g·mol⁻¹≤M_{w}≤480 000 g·mol⁻¹ or 460 000 g·mol⁻¹≤M_{w}≤500 000 g·mol⁻¹;
the copolymer has a polydispersity M̅_{w}/M̅ₙ≤3; and/or
the copolymer has a polydispersity M̅_{w}/M̅ₙ≤2.5, M̅_{w}/M̅ₙ≤2, M̅_{w}/M̅ₙ≤1.8 or M̅_{w}/M̅ₙ≤1.6.

It is a further object of the present invention to provide a process for the synthesis of polymers for pharmaceutical formulations that have dissolution characteristics different from known acrylate copolymers.

This object is achieved by a process comprising the steps of

(a) esterifying an α-hydroxycarboxylic acid selected from the group comprising hydroxy-ethanoic acid, 2-hydroxypropanoic acid, 2-hydroxybutanoic acid, 2-hydroxyisobutanoic acid, 2-hydroxy-2-methyl-3-oxobutanoic acid, phenylhydroxyethanoic acid, 2-hydroxy-4-methylthiobutanoic acid, 2-hydroxybutane-1,4-dioic acid, 2-hydroxypropanedioic acid, 2-hydroxypropane-1,2,3-tricarboxylic acid, hydroxypropane-1,2,3-tricarboxylic acid or 2,3-dihydroxybutanedioic acid having the structure

OH—R≤OH
with
R=—CH₂(C═O)—, R=—CH(CH₃)(C═O)—, R=—CH(CH₂CH₃)(C═O)—, R=—C(CH₃)₂(C═O)—,
R=—C(CH₃)(COCH₃)(C═O)—, R=—CH(Ph)(C═O)—, R=—CH[(CH₂)₂SCH₃](C═O)—,
R=—CH(CH₂COOH)(C═O)—, R=—CH(COOH)(C═O)—, R=—C(CH₂COOH)₂(C═O)—,
R=—CH(COOH)CH(CH₂COOH)(C═O)— or R=—CH(COOH)(CHOH)(C═O)—;
with
acrylic acid ((CH₂)HC—COOH) or methacrylic acid ((CH₂)(CH₃)C—COOH) to give a compound having the structure

Ayl-O—R—OH (Ia)

or

MAyl-O—R—OH (IIa)
in which “Ayl”=acryloyl ((CH₂)HC—CO—) and “MAyl”=methacryloyl ((CH₂)(CH₃)C—CO—);

(b) optionally mono- or polyesterifying the compound (Ia) or (IIa) obtained in step (a) with an α-hydroxycarboxylic acid, in order to obtain a compound of the structure

Ayl-(O—R)ₘ—OH (Ib)
or

MAyl-(O—R)ₘ—OH (IIb)
with 2≤m≤20;

(c) conjugating the compound (Ia), (Ib), (IIa) or (IIb) obtained in step (a) or (b) with a protecting group P, in order to obtain a compound of the structure

Ayl-(O—R)ₙ—OP (Ic)
or

MAyl-(O—R)ₙ—OP (IIc)
with 1≤n≤20;

(d) optionally conjugating acrylic acid or methacrylic acid with the protecting group P in order to obtain protected acrylic acid ((CH₂)HC—COOP) or protected methacrylic acid ((CH₂)(CH₃)C—COOP);

(e) polymerizing the compound (Ic) or (IIc) in a relative molar proportion y with methyl acrylate, methyl methacrylate, ethyl acrylate or ethyl methacrylate in a relative molar proportion x and optionally with protected acrylic acid or protected methacrylic acid in a relative molar proportion z to give a copolymer of the following type:
poly(MAₓ-co-[AS—(O—R)ₙ—OP]_{y}-co-[AS—OP]_{z}),
poly(MMAₓ-co-[AS—(O—R)ₙ—OP]_{y}-co-[AS—OP]_{z}),
poly(EAₓ-co-[AS—(O—R)ₙ—OP]_{y}-co--[AS—OP]_{z}),
poly(EMAₓ-co-[AS—(O—R)ₙ—OP]_{y}-co-[AS—OP]_{z}),
poly(MAₓ-co-[AS—(O—R)ₙ—OP]_{y}-co-[MAS—OP]_{z}),
poly(MMAₓ-co-[AS—(O—R)ₙ—OP]_{y}-co-[MAS—OP]_{z}),
poly(EAₓ-co-[AS—(O—R)ₙ—OP]_{y}-co-[MAS—OP]_{z}),
poly(EMAₓ-co-[AS—(O—R)ₙ—OP]_{y}-co-[MAS—OP]_{z}),
poly(MAₓ-co-[MAS—(O—R)ₙ—OP]_{y}-co-[AS—OP]_{z}),
poly(MMAₓ-co-[MAS—(O—R)ₙ—OP]_{y}-co-[AS—OP]_{z}),
poly(EAₓ-co-[MAS—(O—R)ₙ—OP]_{y}-co-[AS—OP]_{z}),
poly(EMAₓ-co-[MAS—(O—R)ₙ—OP]_{y}-co-[AS—OP]_{z}),
poly(MAₓ-co-[MAS—(O—R)ₙ—OP]_{y}-co-[MAS—OP]_{z}),
poly(MMAₓ-co-[MAS—(O—R)ₙ—OP]_{y}-co-[MAS—OP]_{z}),
poly(EAₓ-co-[MAS—(O—R)ₙ—OP]_{y}-co-[MAS—OP]_{z}),

or
poly(EMAₓ-co-[MAS—(O—R)ₙ—OP]_{y}-co-[MAS—OP]_{z}),
in which MA=methyl acrylate residue (—CH[(C═O)OCH₃]CH₂—), MMA=methyl methacrylate residue (—C(CH₃)[(C═O)OCH₃]CH₂—), EA=ethyl acrylate residue (—CH[(C═O)OCH₂CH₃]CH₂—), EMA=ethyl methacrylate residue (—C(CH₃)[(C═O)OCH₂CH₃]CH₂—); AS=acrylic acid residue (—CH[(C═O)—]CH₂—), MAS=methacrylic acid residue (—C(CH₃)[(C═O)—]CH₂—); 1≤x≤20, 1≤y≤20 and 0≤z≤20; and

(f) deprotecting and hydrolyzing the copolymer obtained in step (e) in order to obtain a copolymer of the following type:
poly(MAₓ-co-[AS—(O—R)ₙ—OH]_{y}-co-[AS—OH]_{z}),
poly(MMAₓ-co-[AS—(O—R)ₙ—OH]_{y}-co-[AS—OH]_{z}),
poly(EAₓ-co-[AS—(O—R)ₙ—OH]_{y}-co-[AS—OH]_{z}),
poly(EMAₓ-co-[AS—(O—R)ₙ—OH]_{y}-co-[AS—OH]_{z}),
poly(MAₓ-co-[AS—(O—R)ₙ—OH]_{y}-co-[MAS—OH]_{z}),
poly(MMAₓ-co-[AS—(O—R)ₙ—OH]_{y}-co-[MAS—OH]_{z}),
poly(EAₓ-co-[AS—(O—R)ₙ—OH]_{y}-co-[MAS—OH]_{z}),
poly(EMAₓ-co-[AS—(O—R)ₙ—OH]_{y}-co-[MAS—OH]_{z}),
poly(MAₓ-co-[MAS—(O—R)ₙ—OH]_{y}-co-[AS—OH]_{z}),
poly(MMAₓ-co-[MAS—(O—R)ₙ—OH]_{y}-co-[AS—OH]_{z}),
poly(EAₓ-co-[MAS—(O—R)ₙ—OH]_{y}-co-[AS—OH]_{z}),
poly(EMAₓ-co-[MAS—(O—R)ₙ—OH]_{y}-co-[AS—OH]_{z})
poly(MAₓ-co-[MAS—(O—R)ₙ—OH]_{y}-co[MAS—OH]_{z}),
poly(MMAₓ-co-[MAS—(O—R)ₙ—OH]_{y}-co[MAS—OH]_{z}),
poly(EAₓ-co-[MAS—(O—R)ₙ—OH]_{y}-co-[MAS—OH]_{z}),
or
poly(EMAₓ-co-[MAS—(O—R)ₙ—OH]_{y}-co-[MAS—OH]_{z}).

Expedient embodiments of the process are characterized in that
in step (a) the α-hydroxycarboxylic acid is protected with a protecting group P before being esterified with acrylic acid or methacrylic acid, and the protecting group P is removed after the esterification;
in the case of single or multiple performance of step (b) the α-hydroxycarboxylic acid is protected with a protecting group P before being esterified with the compound Ayl-(—O—R)_{q}—OH or MAyl-(O—R)_{q}—OH with 1≤q≤m−1, and the protecting group P is removed after the esterification;
the protecting group P is selected from the group comprising benzyl (—CH₂Ph), tert-Butyl (—C(CH₃)₃) and allyl groups;
in step (e) a free-radical polymerization is performed;
in step (e) a RAFT polymerization (reversible addition fragmentation chain transfer polymerization) using a chain transfer reagent is performed;
in step (e) a RAFT polymerization (reversible addition fragmentation chain transfer polymerization) using a chain transfer reagent selected from the group comprising dithioesters and trithiocarbonates is performed;
the deprotection and hydrolysis in step (f) is performed using a catalyst;
the deprotection and hydrolysis in step (f) is performed under elevated pressure in the range from 5 to 100 bar;
n=1, n=2, n=3, n=4, n=5, n=6, n=7, n=8, n=9, n=10, n==11, n==12, n==13, n=14, n=15, n=16, n=17, n=18, n=19 or n=20;
n=1, n=2 or n=3;
x is a real number;
1≤x≤12 or 8≤x≤20;
1≤x≤6, 4≤x≤8, 6≤x≤10, 8≤x≤12, 10≤x≤14, 12≤x≤16, 14≤x≤18 or 16≤x≤20;
1≤x≤3, 2≤x≤4, 3≤x≤5, 4≤x≤6, 5≤x≤7, 6≤x≤8, 7≤x≤9, 8≤x≤10, 9≤x≤11, 10≤x≤12, 11≤x≤13, 12≤x≤14, 13≤x≤15, 14≤x≤16, 15≤x≤17, 16≤x≤18, 17≤x≤19 or 18≤x≤20;
x=1, x=2, x=3, x=4, x=5, x=6, x=7, x=8, x=9, x=10, x=11, x=12, x=13, x=14, x=15, x=16, x=17, x=18, x=19, or x=20;
y is a real number;
1≤y≤12 or 8≤y≤20;
1≤y≤6, 4≤y≤8, 6≤y≤10, 8≤y≤12, 10≤y≤14, 12≤y≤16, 14≤y≤18 or 16≤y≤20;
1≤y≤3, 2≤y≤4, 3≤y≤5, 4≤y≤6, 5≤y≤7, 6≤y≤8, 7≤y≤9, 8≤y≤10, 9≤y≤11, 10≤y≤12, 11≤y≤13, 12≤y≤14, 13≤y≤15, 14≤y≤16, 15≤y≤17, 16≤y≤18, 17≤y≤19 or 18≤y≤20;
y=1, y=2, y=3, y=4, y=5, y=6, y=7, y=8, y=9, y=10, y=11, y=12, y=13, y=14, y=15, y=16, y=17, y=18, y=19 or y=20;
z is a real number;
z=0;
1≤z≤12 or 8≤z≤20;
1≤z≤6, 4≤z≤8, 6≤z≤10, 8≤z≤12, 10≤z≤14, 12≤z≤16, 14≤z≤18 or 16≤z≤20;
1≤z≤3, 2≤z≤4, 3≤z≤5, 4≤z≤6, 5≤z≤7, 6≤z≤8, 7≤z≤9, 8≤z≤10, 9≤z≤11, 10≤z≤12, 11≤z≤13, 12≤z≤14, 13≤z≤15, 14≤z≤16, 15≤z≤17, 16≤z≤18, 17≤z≤19 or 18≤z≤20;
z=1, z=2, z=3, z=4, z=5, z=6, z=7, z=8, z=9, z=10, z=11, z=12, z=13, z=14, z=15, z=16, z=17, z=18, z=19 or z=20;
0.8·x≤y+z≤1.2·x;
0.9·x≤y+z≤1.1·x;
y+z=x;
0.3·x≤y+z≤0.7·x;
0.4·x≤y+z≤0.6·x;
y+z=0.5·x;
y≤z≤6·y, 4·y≤z≤11·y, 8·y≤z≤14·y, 11·y≤z≤17·y or 14·y≤z≤20·y;
y≤z≤3·y, 2·y≤z≤4·y, 3·y≤z≤5·y, 4·y≤z≤6·y, 5·y≤z≤7·y, 6·y≤z≤8·y, 7·y≤z≤9·y, 8·y≤z≤10·y, 9·y≤z≤11·y, 10·y≤z≤12·y, 11·y≤z≤13·y, 12·y≤z≤14·y, 13·y≤z≤15·y, 14·y≤z≤16·y, 15·y≤z≤17·y, 16·y≤z≤18·y, 17·y≤z≤19·y or 18·y≤z≤20·y.

As an alternative to the above “ab initio” synthesis methods, the present invention additionally encompasses processes in which a known acrylate copolymer having a stoichiometric or random repeat unit of the following type:

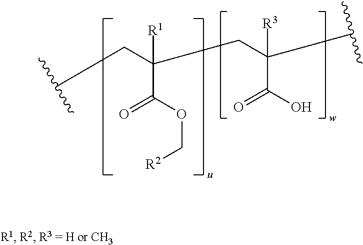

is conjugated with v molar parts of an unprotected α-hydroxycarboxylic acid or α-hydroxycarboxylic acid protected with a protecting group P, selected from the group comprising hydroxyethanoic acid, 2-hydroxypropanoic acid, 2-hydroxybutanoic acid, 2-hydroxyisobutanoic acid, 2-hydroxy-2-methyl-3-oxobutanoic acid, phenylhydroxyethanoic acid, 2-hydroxy-4-methylthiobutanoic acid, 2-hydroxybutane-1,4-dioic acid, 2-hydroxypropanedioic acid, 2-hydroxypropane-1,2,3-tricarboxylic acid, hydroxypropane-1,2,3-tricarboxylic acid or 2,3-dihydroxybutanedioic acid having the structure

OH—R—OH or OH—R—P

where u, v, w are real numbers with
1≤u≤20;
1≤w≤20;
1≤v≤w;

and, when a protected α-hydroxycarboxylic acid is used, the protecting group P is removed in a further process step.

R and P here have the same meaning as set out above, i.e.

R=—CH₂(C═O)—, R=—CH(CH₃)(C═O)—, R=—CH(CH₂CH₃)(C═O)—, R=—C(CH₃)₂(C═O)—, R=—C(CH₃)(COCH₃)(C═O)—, R=—CH(Ph)(C═O)—, R=—CH[(CH₂)₂SCH₃](C═O)—, R=—CH(CH₂COOH)(C═O)—, R=—CH(COOH)(C═O)—, R=—C(CH₂COOH)₂(C═O)—, R=—CH(COOH)CH(CH₂COOH)(C═O)— or R=—CH(COOH)(CHOH)(C═O)—; and P=benzyl (—CH₂Ph), P=tert-butyl (—C(CH₃)₃) or P=allyl group.

Expedient embodiments of the process for modifying known acrylate copolymers with unprotected or protected α-hydroxycarboxylic acid are characterized in that:
glycolic acid is used as α-hydroxycarboxylic acid;
lactic acid is used as α-hydroxycarboxylic acid;
0.8·u≤w≤1.2·u;
0.3·u≤w≤0.7·u;
v≤w≤6·v, 5·v≤w≤11·v, 8·v≤w≤14·v, 11·v≤w≤17·v or 14·v≤w≤20·v;
v≤w≤3·v, 2·v≤w≤4·v, 3·v≤w≤5·v, 4·v≤w≤6·v, 5·v≤w≤7·v, 6·v≤w≤8·v, 7·v≤w≤9·v, 8·v≤w≤10·v, 9·v≤w≤11·v, 10·v≤w≤12·v, 11·v≤w≤13·v, 12·v≤w≤14·v, 13·v≤w≤15·v, 14·v≤w≤16·v, 15·v≤w≤17·v, 16·v≤w≤18·v, 17·v≤w≤19·v or 18·v≤w≤20·v;
DIPC (diisopropylcarbodiimide) is used as coupling reagent;
DMAP (4-(N,N-dimethylamino)pyridine) is used as catalyst; and/or
the reaction is conducted in an organic solvent, such as benzene, dioxane or DMF.

This polymer-analogous method can be performed for copolymers of any molecular weight. Eudragit L100 has a molecular weight of about 125 000 g/mol and Eudragit L100-55 has a molecular weight of about 320 000 g/mol. These copolymers are prepared by means of suspension or emulsion polymerization and can be subjected to polymer-analogous modification in an additional reaction step, as described above.

The invention further relates to copolymers preparable by one of the processes described above.

The invention further relates to the use of the above-described copolymers for the production of pharmaceutical formulations, tablet or capsule coatings.

The abbreviations “AS” and “MAS” used in the context of the present invention for an acrylic acid residue AS=—CH[(C═O)—]CH₂— and a methacrylic acid residue MAS=—C(CH₃)[(C═O)—]CH₂— and monomers containing these residues have the following meaning:

[AS—OH]=—CH[(C═O)—OH]CH₂—;

[MAS—OH]=—C(CH₃)[(C═O)—OH]CH₂—;

[AS—(O—R)ₙ—OH]=—CH[(C═O)—(O—R)ₙ—OH]CH₂—;

[MAS—(O—R)ₙ—OH]=—C(CH₃)[(C═O)—(O—R)ₙ—OH]CH₂—.

The α-hydroxycarboxylic acids used in the context of the present invention are listed in table 3 below:

TABLE 3
α-Hydroxycarboxylic acids

| α-Hydroxycarboxylic acid | Structure | Residue R |
|---|---|---|
| Hydroxyethanoic acid (glycolic acid) | 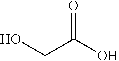 | —CH₂(C═O)— |
| 2-Hydroxypropanoic acid lactic acid | 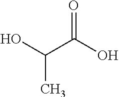 | —CH(CH₃)(C═O)— |
| 2-Hydroxybutanoic acid | 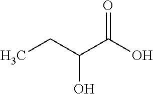 | —CH(CH₂CH₃)(C═O)— |
| 2-Hydroxyisobutanoic acid | 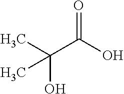 | —C(CH₃)₂(C═O)— |
| 2-Hydroxy-2-methyl-3- oxobutanoic acid | 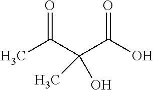 | —C(CH₃)(COCH₃)(C═O)— |
| Phenylglycolic acid (mandelic acid) | 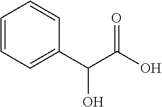 [SMILES: O=C(O)C(O)c1ccccc1] | —CH(Ph)(C═O)— |
| 2-Hydroxy-4-methylthio- butanoic acid (methionine) | 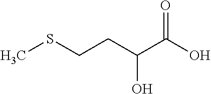 | —CH[(CH₂)₂SCH₃](C═O)— |
| 2-Hydroxybutane-1,4-dioic acid (malic acid) | 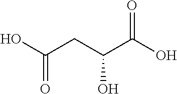 | —CH(CH₂COOH)(C═O)— |
| 2-Hydroxypropanedioic acid (tartronic acid) | 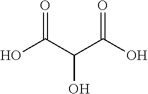 | —CH(COOH)(C═O)— |
| 2-Hydroxypropane-1,2,3- tricarboxylic acid (citric acid) | 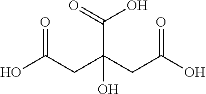 [SMILES: O=C(O)CC(O)(CC(=O)O)C(=O)O] | —C(CH₂COOH)₂(C═O)— |
| Hydroxypropane-1,2,3- tricarboxylic acid (isocitric acid) | 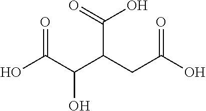 [SMILES: O=C(O)CC(C(=O)O)C(O)C(=O)O] | —CH(COOH)CH(CH₂COOH)(C═O)— |
| 2,3-Dihydroxybutanedioic acid (tartaric acid) | 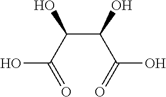 | —CH(COOH)(CHOH)(C═O)— |

Table 3 specifies, for each of the α-hydroxycarboxylic acids, a residue R from which the side arms of the copolymers of the invention are essentially formed. A side arm comprises 1 to 20 residues R.

In a preferred embodiment of the invention, the stoichiometric or statistical repeat unit of the acrylate copolymers has the following structure:

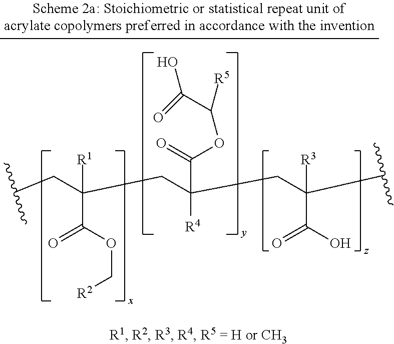

In the repeat unit of scheme 2a, x, y, z denote real numbers that fulfill the following conditions:
1≤x≤20, 1≤y≤20, 1≤z≤20;
0.8·x≤y+z≤1.2·x or 0.3·x≤y+z≤0.7·x;
y≤z≤6·y, 5·y≤z≤11·y, 8·y≤z≤14·y, 11·y≤z≤17·y or 14·y≤z≤20·y; and/or
y≤z≤3·y, 2·y≤z≤4·y, 3·y≤z≤5·y, 4·y≤z≤6·y, 5·y≤z≤7·y, 6·y≤z≤8·y, 7·y≤z≤9·y, 8·y≤z≤10·y, 9·y≤z≤11·y, 10·y≤z≤12·y, 11·y≤z≤13·y, 12·y≤z≤14·y, 13·y≤z≤15·y, 14·y≤z≤16·y, 15·y≤z≤17·y, 16·y≤z≤18·y, 17·y≤z≤19·y or 18·y≤z≤20·y.

The R¹, R², R³, R⁴, R⁵ residues are independently —H or —CH₃.

In particularly preferred embodiments of the invention, the R³ and R⁵ residues in the repeat unit shown in scheme 2a are the same, i.e. R³=R⁵=—H or R³=R⁵=—CH₃, Acrylate copolymers according to scheme 2a with R³=R⁵ are preferably synthesized by a simple process having the following steps:

(a′) copolymerizing methyl acrylate, methyl methacrylate, ethyl acrylate or ethyl methacrylate with unprotected or protected acrylic acid or methacrylic acid;

(b′) if protected acrylic acid or protected methacrylic acid has been used in step (a′), deprotecting the acrylate copolymer obtained in step (a′);

(c′) esterifying the acrylate copolymer obtained in step (a′) or (b′) with protected glycolic acid or protected lactic acid; and

(d′) deprotecting the acrylate copolymer obtained in step (c′).

The invention is illustrated in detail below by examples, where the indices n, x and y have a definition independent of the above description and of the claims.

### EXAMPLE 1

### Synthesis Strategy for Acrylate Copolymers Containing α-Hydroxycarboxylic Acid Residues

α-Hydroxycarboxylic acids are bifunctional. Therefore, direct esterification of an α-hydroxycarboxylic acid with acrylic acid or methacrylic acid would form a mixture of different oligomers. In order to prevent this, the acid group is reversibly protected. Suitable protecting groups for this purpose include benzyl, tert-butyl or allyl groups, since they are easy to introduce and stable to the reaction conditions in subsequent process steps. The protected α-hydroxycarboxylic acid can be conjugated with acrylic acid or methacrylic acid in a Steglich esterification to give a monomer. The resultant monomer is copolymerized with methyl methacrylate or ethyl acrylate, and then the protecting group is removed. This synthesis strategy is illustrated in scheme 3.

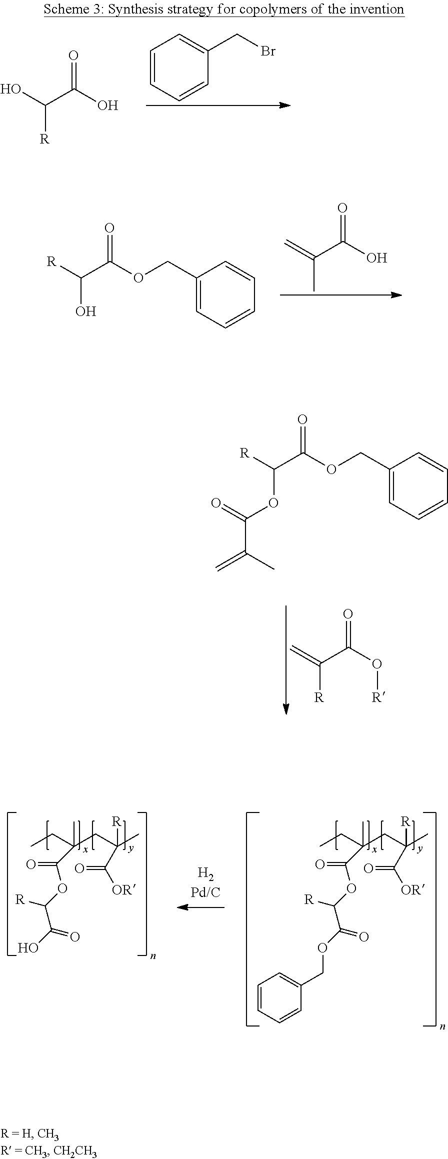

After the polymerization, the protecting group is removed by palladium/charcoal-catalyzed reduction with hydrogen.

### EXAMPLE 2

### Modification of Acrylate Copolymers

Scheme 4 illustrates the synthesis strategy for the modification of acrylate copolymers with a protected α-hydroxycarboxylic acid, for example with benzyl-protected glycolic acid (hydroxyethanoate benzyl or “Gly-Bn”).

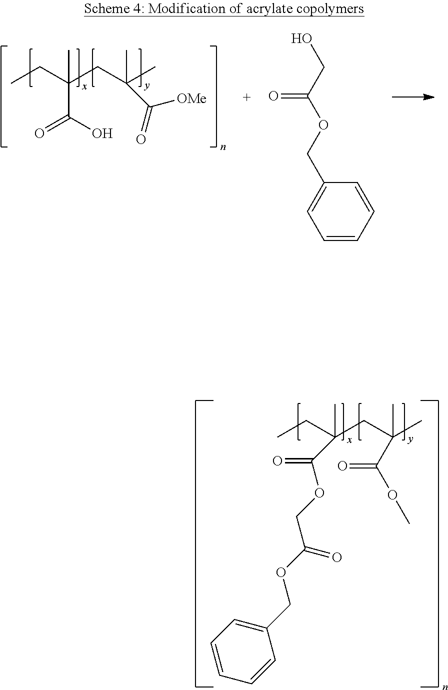

### EXAMPLE 3

### Principle of Steglich Esterification

Scheme 5 illustrates the principle of Steglich esterification which is employed in the context of the present invention for the simple or iterative conjugation of acrylic acid or methacrylic acid with a protected α-hydroxycarboxylic acid.

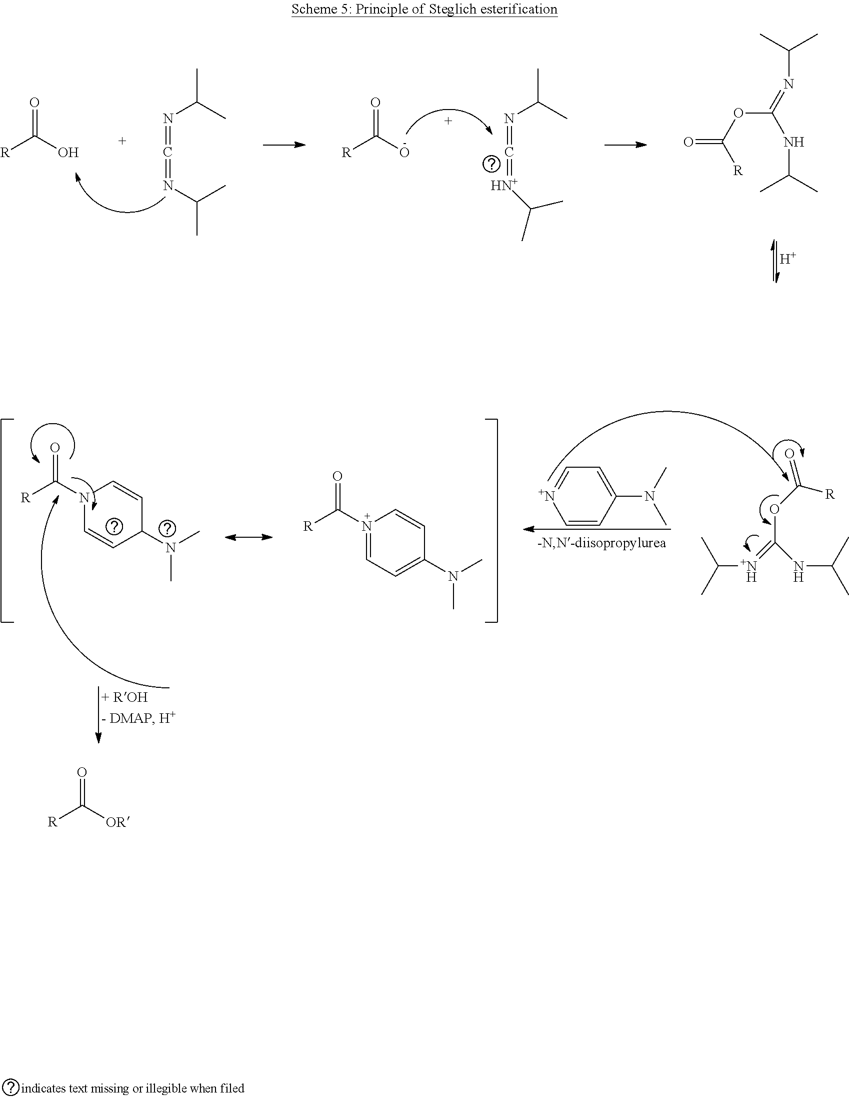

In general, in an esterification reaction, an organic acid is reacted with an alcohol to give an ester. Owing to the low carbonyl activity of the acid, the reaction with the alcohol is slow. With increasing space filling by reactants, the reaction rate decreases. Carbonyl activity is generally increased using carbonyl chlorides and carboxylic anhydrides. However, given the reactants used in the present invention, carbonyl chlorides are unsuitable.

In the present invention, preference is given to employing the principle of Steglich esterification. Steglich esterification gives good yields under gentle reaction conditions. The coupling reagent used is appropriately DIPC (diisopropylcarbodiimide), and the catalyst DMAP (4-(N,N-dimethylamino)pyridine). The reaction mechanism is shown in scheme 5. DIPC together with the acid first forms an O-acylisourea, the carbonyl activity of which is comparable to that of the anhydride of the acid. DMAP, which is a stronger nucleophile than the alcohol used, together with the acylisourea, forms an N,N′-diisopropylurea and a reactive amide, which is also referred to as “active ester”. The latter together with the alcohol forms one of the esters envisaged in accordance with the invention, and DMAP, which is available thereafter as acyl transfer reagent.

### EXAMPLE 4

### Principle of RAFT Polymerization

The polymers of the invention are appropriately copolymerized by the RAFT principle shown in scheme 6. RAFT polymerization is a method of synthesis of polymers having a narrow molecular weight distribution. For this purpose, what is called a chain transfer reagent is added to the reaction solution in addition to solvent, monomer and initiator. This reacts with, and inactivates, the free-radical chain in a kinetic equilibrium. Suitable chain transfer reagents are especially dithioesters and trithiocarbonates.

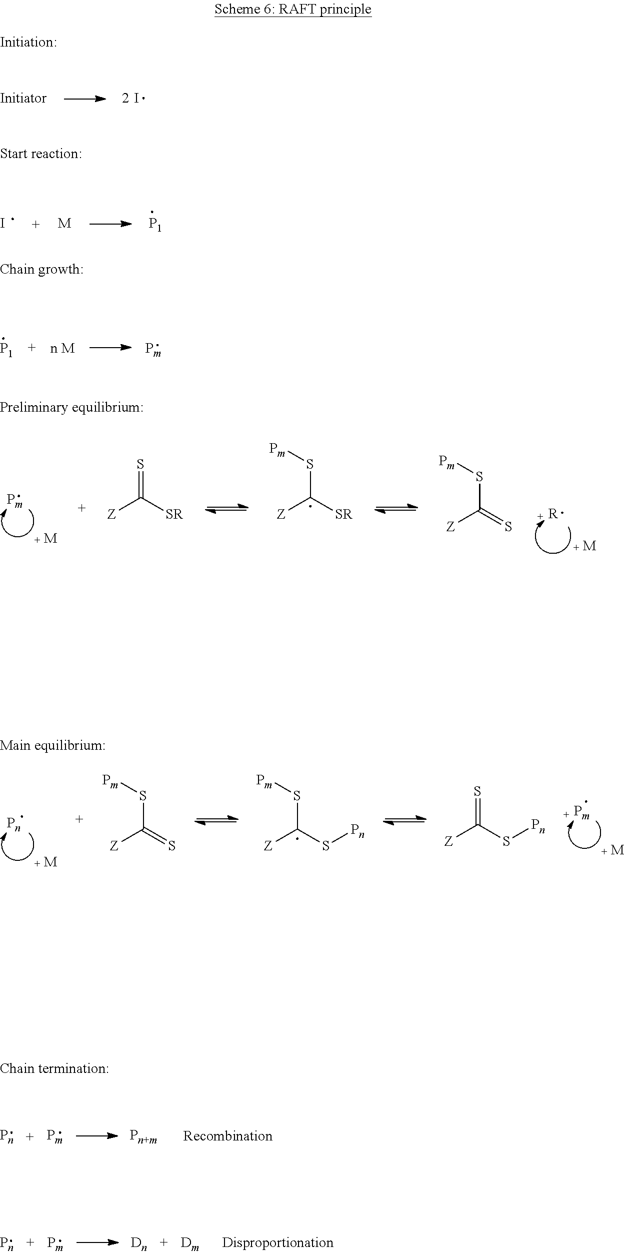

Initiators used are conventional initiators such as AlBN (azoisobutyronitrile) or dibenzoyl peroxide. After they have split into reactive free radicals, these react with the monomer used. After the start reaction or initialization, the chain grows according to the free-radical mechanism. If the free-radical end of a growing molecule chain meets a chain transfer reagent, an adduct radical is formed, which is in a temporary equilibrium with the polymer dithioester and the free radical R. The free radical R can initiate the formation of a new free-radical chain. The primary RAFT equilibrium is between the polymer dithioester and a further free-radical chain. The adduct radical does not react with the monomer and is referred to as a “sleeping” species. This greatly reduces the concentration of active free radicals. On account of the kinetic equilibrium, all chains have the same average growth time and attain the same degree of polymerization. The polydispersity achieved in RAFT polymerization is in the range from 1.1 to 1.3.

The polymers of the invention are preferably synthesized by means of RAFT polymerization, in order to obtain a low polydispersity, associated with defined dissolution characteristics. Moreover, the chain transfer reagent used in the RAFT polymerization enables the introduction of a group having an NMR signature. With the aid of the NMR signature, it is possible to determine the total number of monomers in the polymer chain.

### EXAMPLE 5

### Hydroxyethanoate Benzyl Ester (Gly-Bn)

### Reaction Equation

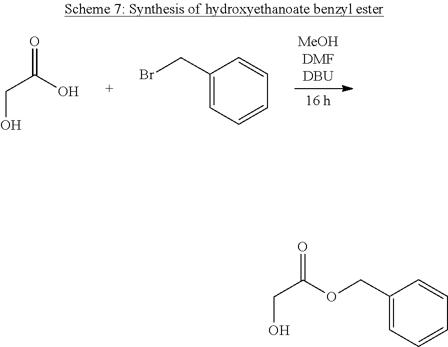 [SMILES: O=C(O)CO ; BrCc1ccccc1 ; O=C(CO)OCc1ccccc1]

### Reaction

TABLE 4
Reaction table for the synthesis of hydroxyethanoate benzyl ester

| Substance | M/(g/mol) | n/(mol) | Eq. | m/(g) | ρ/(g/mL) | V/(mL) |
|---|---|---|---|---|---|---|
| Glycolic | 76.05 | 0.3942 | 1.0 | 29.98 | — | — |
| acid | | | | | | |
| DBU | 152.24 | 0.3940 | 1.0 | 59.98 | 1.02 | 58.8 |
| Benzyl | 171.04 | 0.3949 | 1.0 | 67.54 | 1.44 | 46.9 |
| bromide | | | | | | |
| Methanol | 32.04 | — | — | 118.50 | 0.79 | 150.0 |
| DMF | 73.10 | — | — | 228.00 | 0.95 | 240.0 |

### Procedure

Glycolic acid was dissolved with 150 mL of methanol in a 250 mL one-neck flask. 1,8-Diazabicyclo[5.4.0]undec-7-ene was added dropwise thereto with a syringe while stirring. After stirring for 30 minutes, methanol was removed at 50° C. under reduced pressure. The resultant oily liquid was dissolved in 240 mL of TV,TV-dimethylformamide and cooled to 15° C., and benzyl bromide was slowly added with a dropping funnel while stirring. This solution was stirred at room temperature for 18 hours. 250 mL of ethyl acetate and 400 mL of water were added to the solution. The aqueous phase was then extracted by shaking four times with 150 mL each time of ethyl acetate. The combined organic phases were washed with 150mL of water, three times with 100 mL of 5% citric acid and twice with 150 mL of a saturated sodium chloride solution, and then dried with aqueous sodium sulfate. The ethyl acetate was removed at 50° C. under reduced pressure. The hydroxyethanoate benzyl ester was purified by fractional distillation at 1·10⁻³ bar and 98° C.

### Characterization

Appearance: colorless liquid

Yield: 51.11 g, 0.3076 mol, 78%

Boiling point: 98° C. at 1·10⁻³ bar

M=166.17 g/mol

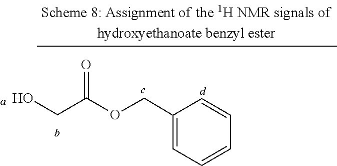 [SMILES: O=C(CO)OCc1ccccc1]

¹H NMR: (400 MHz; CDCl₃): δ[ppm]=2.36 (s, 1H, Ha), 4.20 (s, 2H, Hb), 5.24 (s, 2H, Hc), 7.34-7.40 (m, 5H, Hd)

### EXAMPLE 6

### (S)-2-Hydroxypropionate Benzyl Ester (L-La-Bn)

### Reaction Equation

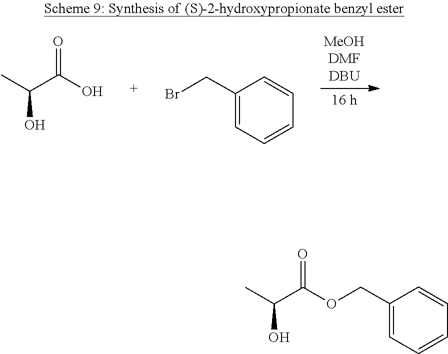

### Reaction

TABLE 5
Reaction table for the synthesis of (S)-2-
hydroxypropionate benzyl ester (L-La-Bn)

| Substance | M/(g/mol) | n/(mol) | Eq. | m/(g) | ρ/(g/mL) | V/(mL) |
|---|---|---|---|---|---|---|
| L-lactic | 90.08 | 0.2629 | 1.0 | 23.68 | — | — |
| acid | | | | | | |
| DBU | 152.24 | 0.2626 | 1.0 | 39.98 | 1.02 | 39.2 |
| Benzyl | 171.04 | 0.2635 | 1.0 | 45.07 | 1.44 | 31.3 |
| bromide | | | | | | |
| Methanol | 32.04 | — | — | 118.50 | 0.79 | 150.0 |
| DMF | 73.10 | — | — | 142.50 | 0.95 | 150.0 |

### Procedure

The procedure was analogous to the synthesis of hydroxyethanoate benzyl ester.

### Characterization

Appearance: colorless liquid

Yield: 34.66 g, 0.1923 mol, 71.6%

Boiling point: 96° C. at 1·10⁻³ bar

M=180.20 g/mol

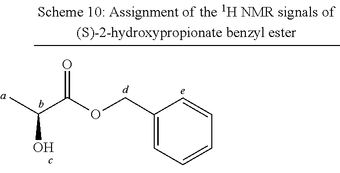

¹H NMR: (400 MHz; CDCl₃): δ[ppm]=1.44 (d,3H, Ha), 2.83 (s, 1H, Hc), 4.32 (q, 1H, Hb), 5.21 (s, 2H, Hd), 7.33-7.40 (m, 5H, He)

### EXAMPLE 7

### 2-Hydroxypropionate Benzyl Ester (D,L-La-Bn)

### Reaction Equation

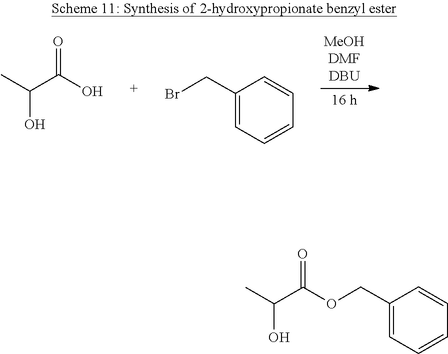

### Reaction

TABLE 6
Reaction table for the synthesis of
2-hydroxypropionate benzyl ester

| Substance | M/(g/mol) | n/(mol) | Eq. | m/(g) | ρ/(g/mL) | V/(mL) |
|---|---|---|---|---|---|---|
| D,L-lactic | 90.08 | 0.111 | 1.0 | 10.00 | 1.21 | 8.26 |
| acid | | | | | | |
| DBU | 152.24 | 0.111 | 1.0 | 16.90 | 1.02 | 16.57 |
| Benzyl | 171.04 | 0.111 | 1.0 | 18.99 | 1.44 | 13.19 |
| bromide | | | | | | |
| Methanol | 32.04 | — | — | 118.50 | 0.79 | 150.00 |
| DMF | 73.10 | — | — | 142.50 | 0.95 | 150.00 |

### Procedure

The procedure was analogous to the synthesis of hydroxyethanoate benzyl ester.

### Characterization

Appearance: colorless liquid

Yield: 13.02 g, 0.0723 mol, 65.1%

Boiling point: 91° C. at 4.8·10⁻³ bar

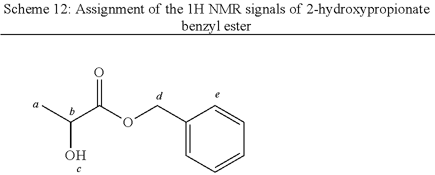

¹H NMR: (400 MHz; CDCl₃): δ[ppm]=1.44 (d,3H, Ha), 2.77 (s, 1H, Hc), 4.32 (q, 1H, Hb), 5.22 (s, 2H, Hd), 7.32-7.41 (m, 5H, He)

### EXAMPLE 8

### 2-Methacryloyloxyethanoate Benzyl Ester (MAylO-Gly-Bn)

### Reaction Equation

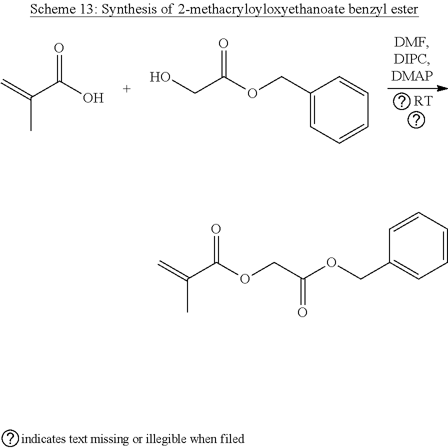

### Reaction

TABLE 7
Reaction table for the synthesis of 2-
methacryloyloxyethanoate benzyl ester

| Substance | M/(g/mol) | n/(mol) | Eq. | m/(g) | ρ/(g/mL) | V/(mL) |
|---|---|---|---|---|---|---|
| Gly-Bn | 166.17 | 0.0617 | 1 | 10.00 | — | — |
| Meth- | 86.09 | 0.0619 | 1.1 | 5.70 | 1.02 | 5.59 |
| acrylic | | | | | | |
| acid | | | | | | |
| DIPC | 126.20 | 0.0902 | 1.5 | 11.39 | 0.81 | 14.06 |
| DMAP | 122.17 | 0.0060 | 0.1 | 0.74 | — | — |
| DMF | 73.10 | — | — | 57.00 | 0.95 | 60.00 |

### Procedure

Gly-Bn, methacrylic acid and DMAP were transferred in a 500 mL UV-opaque one-neck flask and brought into solution with 35 mL. Subsequently, the solution was cooled to 0° C. in an ice bath, and DIPC dissolved in 15 mL of DMF was added dropwise with a pressure-equalizing dropping funnel while stirring, and rinsed in with 10 mL of DMF. During the reaction, a precipitate was formed. After the addition, the cooling was removed and the solution was stirred for 5 days.

For workup of the product, the precipitate was filtered off, and 100 mL of ethyl acetate and 100 mL of water were added to the yellowish solution. The aqueous phase was extracted by shaking three times with 150 mL each time of ethyl acetate. The combined organic phases were washed with 150 mL of water and twice with 150 mL of a saturated sodium chloride solution. Drying was effected with magnesium sulfate, and 0.1 g of BHT was added as stabilizer. The solvent was removed at 50° C. under reduced pressure. This resulted in precipitation of a colorless precipitate. The solution was stored at 26° C. overnight. The precipitate was filtered off and washed with ice-cold ethyl acetate. The solvent was removed again at 50° C. under reduced pressure. This was followed by column chromatography of the product (EtAc:PE, 1:5).

### Characterization

Appearance: colorless liquid

Yield: 8.45 g, 0.0361 mol, 58.5%

M=234.25 g/mol

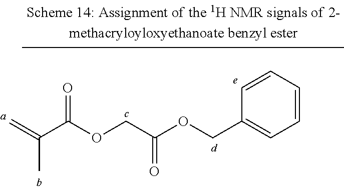 [SMILES: C=C(C)C(=O)OCC(=O)OCc1ccccc1]

¹H NMR: (400 MHz; CDCl₃): δ[ppm]=1.99 (m, 3H, Hb), 4.73 (s, 2H, Hc), 5.21 (s, 2H, Hd), 5.66 (m, 1H, Ha), 6.23 (m, 1H, Ha), 7.33-7.38 (m, 5H, He)

### EXAMPLE 9

### (S)-2-Methacryloyloxypropionate Benzyl Ester (MAylO-L-La-Bn)

### Reaction Equation

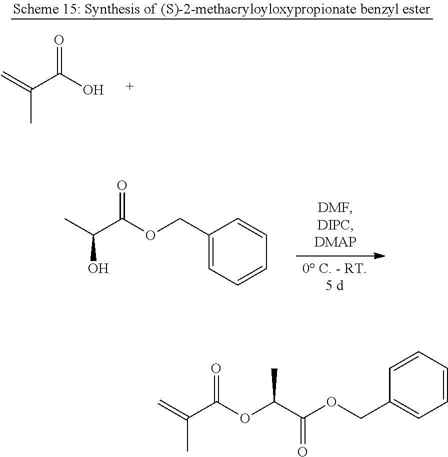

### Reaction

TABLE 8
Reaction table for the synthesis of (S)-
2-methacryloyloxy-propionate benzyl ester

| Substance | M/(g/mol) | n/(mol) | Eq. | m/(g) | ρ/(g/mL) | V/(mL) |
|---|---|---|---|---|---|---|
| L-La-Bn | 180.20 | 0.192 | 1.0 | 34.66 | — | — |
| Meth- | 86.09 | 0.211 | 1.1 | 18.21 | 1.02 | 17.85 |
| acrylic | | | | | | |
| acid | | | | | | |
| DIPC | 126.20 | 0.288 | 1.5 | 36.41 | 0.81 | 44.95 |
| DMAP | 122.17 | 0.019 | 0.1 | 2.35 | — | — |
| DMF | 73.10 | — | — | 57.00 | 0.95 | 60.00 |

### Procedure

The procedure was analogous to the synthesis of 2-methacryloyloxyethanoate benzyl ester.

### Characterization

Appearance: colorless liquid

Yield: 28.31 g, 0.1140 mol, 59.4%

M=248.28 g/mol

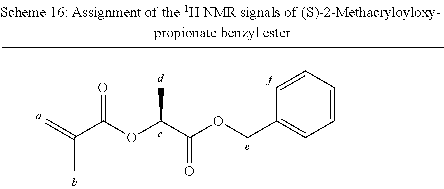 [SMILES: C=C(C)C(=O)O[C@@H](C)C(=O)OCc1ccccc1]

¹H NMR: (400 MHz; CDCl₃): δ[ppm]=1.55 (d, 3H, Hd), 1.97 (m, 3H, Hb), 5.18 (q, 1H, Hc), 5.20 (s, 2H, He), 5.63 (m, 1H, Ha), 6.20 (m, 1H, Ha), 7.32-7.38 (m, 5H, Hf)

### EXAMPLE 10

### 2-Methacryloyloxypropionate Benzyl Ester (MAylO-D,L-La-Bn)

### Reaction Equation

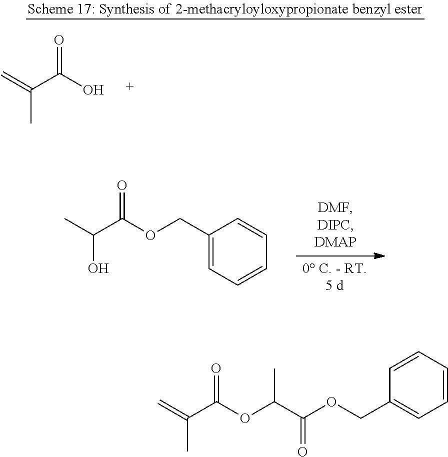

### Reaction

TABLE 9
Reaction table for the synthesis of 2-
methacryloyloxypropionate benzyl ester

| Substance | M/(g/mol) | n/(mol) | Eq. | m/(g) | ρ/(g/mL) | V/(mL) |
|---|---|---|---|---|---|---|
| D,L-La- | 180.20 | 0.072 | 1.0 | 13.00 | — | — |
| Bn | | | | | | |
| Meth- | 86.09 | 0.079 | 1.1 | 6.831 | 1.02 | 6.697 |
| acrylic | | | | | | |
| acid | | | | | | |
| DIPC | 126.20 | 0.108 | 1.5 | 13.656 | 0.81 | 16.859 |
| DMAP | 122.17 | 0.007 | 0.1 | 0.881 | — | — |
| DMF | 73.10 | — | — | 57.00 | 0.95 | 60.00 |

### Procedure

The procedure was analogous to the synthesis of 2-methacryloyloxyethanoate benzyl ester.

### Characterization

Appearance: colorless liquid

Yield: 8.19 g, 0.0330 mol, 45.8%

M=248.28 g/mol

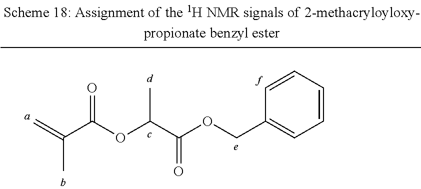 [SMILES: C=C(C)C(=O)OC(C)C(=O)OCc1ccccc1]

¹H NMR: (400 MHz; CDCl₃): δ[ppm]=1.54 (d, 3H, Hd), 1.97 (m, 3H, Hb), 5.18 (q, 1H, Hc), 5.20 (s, 2H, He), 5.63 (m, 1H, Ha), 6.21 (m, 1H, Ha), 7.32-7.37 (m, 5H, Hf).

### EXAMPLE 11

### Copolymerizations

### DMPA Initiator

### Reaction Equation

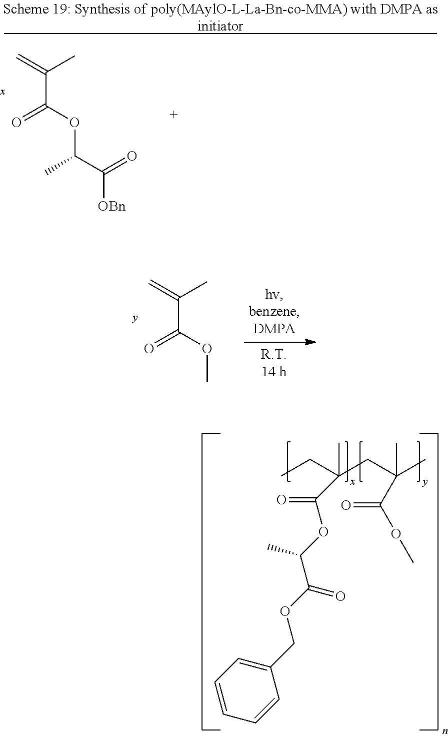

### Reaction

TABLE 10
Reaction table for the copolymerization of
MAylO-L-La-Bn with MMA in a ratio of 1:1

| Substance | M/(g/mol) | n/(mol) | Eq. | m/(g) | ρ/(g/mL) | V/(mL) |
|---|---|---|---|---|---|---|
| MAylO-L-La- | 248.28 | 0.0012 | 1.00 | 0.30 | — | — |
| Bn | | | | | | |
| MMA | 100.12 | 0.0012 | 1.00 | 0.12 | 0.94 | 0.13 |
| Benzene | 78.11 | 0.027 | — | 2.11 | 0.88 | 2.40 |

TABLE 11
Variations of the initiator concentrations in
percent by mass for DMPA and the corresponding
yields, at a monomer concentration of 1 mol/L

| Run | Percent by mass | m/(g) | Yield |
|---|---|---|---|
| 1 | 2 wt % | 0.0084 | 34% |
| 2 | 3 wt % | 0.0126 | 43% |
| 3 | 4 wt % | 0.0168 | 55% |
| 4 | 5 wt % | 0.0210 | 70% |

TABLE 12
Variations in the amounts of benzene used; 0.021
g of DMPA was used, corresponding to 5 wt %

| Run | Concentration | Benzene/mL | Yield |
|---|---|---|---|
| 5 | 1 mol/L | 2.4 | 82 |
| 6 | 1.5 mol/L | 1.8 | 89 |
| 7 | 2 mol/L | 1.2 | 99 |

### Procedure

The MAylO-L-La-Bn and methyl methacrylate monomers were columned through neutral alumina and initially charged in a Schlenk tube. Subsequently, the initiator dissolved in benzene was added, and the tube was closed with a septum. This solution was subjected to three freeze-pump procedures. The Schlenk tube was positioned in front of a UV lamp for 14 h. The polymers were precipitated twice in ice-cold petroleum ether and dried on the Schlenk apparatus.

### Characterization

Appearance: colorless solid

TABLE 13
DMF-GPC data of the polymers prepared with various monomer
concentrations; the standard used was toluene and the
calibration curve was created with polyethylene glycol

| Run | Mₙ | Ð |
|---|---|---|
| 5 | 2570 g/mol | 1.96 |
| 6 | 2950 g/mol | 1.75 |
| 7 | 3250 g/mol | 1.89 |

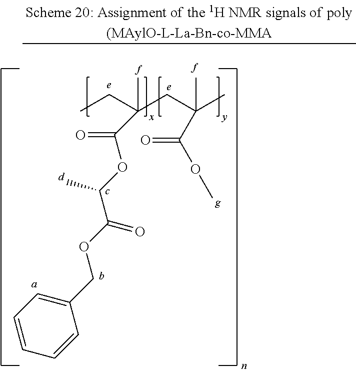

¹H NMR: (400 MHz; DMSO-d₆): δ[ppm]=0.66-1.20 (6H, Hf), 1.32-1.48 (3H, Hd), 1.62-2.08 (4H, He), 3.43-3.62 (3H, Hg), 4.85-5.07 (1H, Hc), 5.07-5.24 (2H, Hb), 7.28-7.41 (5H, Ha)

### AlBN initiator

### Reaction Equation

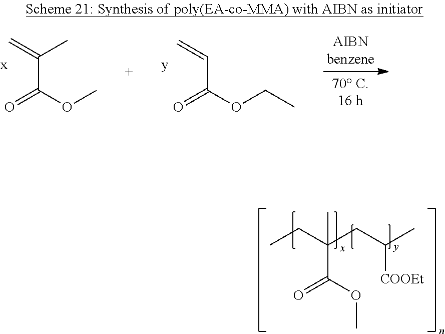

### Reaction

TABLE 14
Reaction table of the copolymerization of EA with MMA in
a ratio of 1:1, at a monomer concentration of 2.5 mol/L

| Substance | M/(g/mol) | n/(mol) | Eq. | m/(g) | ρ/(g/mL) | V/(mL) |
|---|---|---|---|---|---|---|
| MMA | 100.12 | 0.002 | 1 | 0.20 | 0.94 | 0.21 |
| EA | 100.12 | 0.002 | 1 | 0.20 | 0.92 | 0.22 |
| Benzene | 78.11 | 0.023 | — | 1.81 | 0.88 | 1.60 |

TABLE 15
Variations of initiator concentrations in percent
by mass for AlBN and the corresponding yields

| Run | Percent by mass | m/(g) | Yield |
|---|---|---|---|
| 1 | 3 wt % | 0.012 | >99% |
| 2 | 4 wt % | 0.016 | >99% |
| 3 | 5 wt % | 0.020 | >99% |
| 4 | 6 wt % | 0.024 | >99% |

TABLE 16
Variations in the amounts of benzene used; 0.016 g of
AlBN was used as initiator, corresponding to 4 wt %

| Run | Concentration | Benzene/mL | Yield |
|---|---|---|---|
| 5 | 1.5 mol/L | 2.6 | 75% |
| 6 | 2.0 mol/L | 2.0 | 64% |
| 7 | 2.5 mol/L | 1.6 | 70% |

### Procedure

The ethyl acrylate and methyl methacrylate monomers were columned through neutral alumina and hence freed of the stabilizer. They were subsequently initially charged in a Schlenk tube. The 2,2-azobis(2-methylpropionitrile) initiator was dissolved in benzene and transferred into the Schlenk tube. The Schlenk tube was closed with a glass stopper and subjected to three freeze-pump procedures. The solution was then heated to 70° C. while stirring for 16 h. The polymers were precipitated twice in ice-cold petroleum ether and dried on the Schlenk apparatus.

### Characterization

Appearance: colorless solid

Characterization by means of ¹H NMR was dispensed with, since it was only the yields and mass distributions that were of relevance for the study of the initiator system.

TABLE 17
DMF-GPC data for the study of the AlBN initiator system

| Run | Mₙ | Ð |
|---|---|---|
| 1 | 14 530 g/mol | 2.20 |
| 2 | 34 520 g/mol | 1.35 |
| 3 | 13 490 g/mol | 1.95 |
| 4 | 22 120 g/mol | 1.76 |
| 5 | 21 280 g/mol | 1.61 |
| 6 | 30 810 g/mol | 1.39 |
| 7 | 34 520 g/mol | 1.35 |

### EXAMPLE 12

### Poly(MAylO-L-La-Bn-co-MMA)

### Reaction Equation

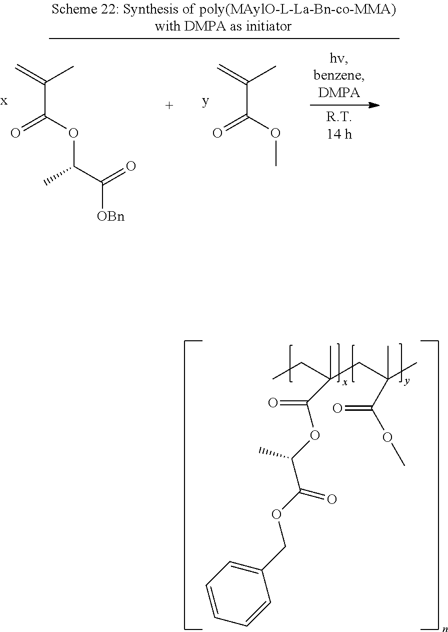

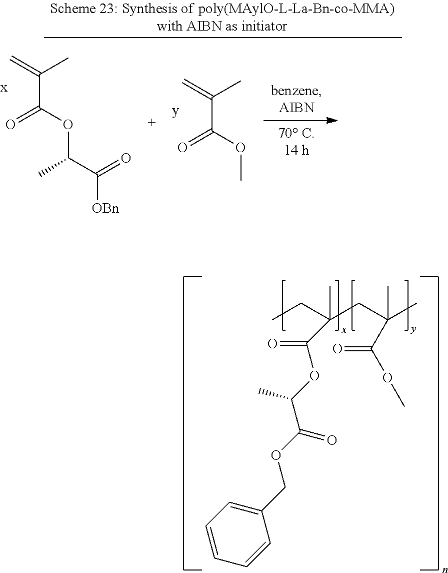

### Reaction

TABLE 18
Reaction table for the copolymerization of
MAylO-L-La-Bn with MMA in a ratio of 1:1

| Substance | M/(g/mol) | n/(mol) | Eq. | m/(g) | ρ/(g/mL) | V/(mL) |
|---|---|---|---|---|---|---|
| MAylO-L- | 248.28 | 0.0032 | 1.00 | 0.800 | — | — |
| La-Bn | | | | | | |
| MMA | 100.12 | 0.0032 | 1.00 | 0.322 | 0.94 | 0.34 |
| Benzene | — | — | — | 5.63 | 0.88 | 6.40 |
| DMPA | 256.29 | 0.00022 | 5% wt | 0.0561 | — | — |

TABLE 19
Reaction table for the copolymerization of
MAylO-L-La-Bn with MMA in a ratio of 1:2

| Substance | M/(g/mol) | n/(mol) | Eq. | m/(g) | ρ/(g/mL) | V/(mL) |
|---|---|---|---|---|---|---|
| MAylO-L- | 248.28 | 0.0012 | 1.00 | 0.300 | — | — |
| La-Bn | | | | | | |
| MMA | 100.12 | 0.0024 | 2.00 | 0.242 | 0.94 | 0.26 |
| Benzene | — | — | — | 3.17 | 0.88 | 3.60 |
| DMPA | 256.29 | 0.00011 | 5% wt | 0.0274 | — | — |

TABLE 20
Reaction table for the copolymerization of
MAylO-L-La-Bn with MMA in a ratio of 2:1

| Substance | M/(g/mol) | n/(mol) | Eq. | m/(g) | ρ/(g/mL) | V/(mL) |
|---|---|---|---|---|---|---|
| MAylO-L- | 248.28 | 0.0012 | 2.00 | 0.300 | — | — |
| La-Bn | | | | | | |
| MMA | 100.12 | 0.0006 | 1.00 | 0.060 | 0.94 | 0.06 |
| Benzene | — | — | — | 1.58 | 0.88 | 1.80 |
| DMPA | 256.29 | 0.00007 | 5% wt | 0.0180 | — | — |

TABLE 21
Reaction table for the copolymerization of MAylO-L-La-Bn
with MMA in a ratio of 1:1 with AlBN as initiator

| Substance | M/(g/mol) | n/(mol) | Eq. | m/(g) | ρ/(g/mL) | V/(mL) |
|---|---|---|---|---|---|---|
| MAylO-L- | 248.28 | 4.43 · | 1.00 | 1.100 | — | — |
| La-Bn | | 10⁻³ | | | | |
| MMA | 100.12 | 4.43 · | 1.00 | 0.4435 | 0.94 | 0.47 |
| | | 10⁻³ | | | | |
| Benzene | 78.11 | 0.039 | — | 3.1152 | 0.88 | 3.54 |
| AlBN | 164.21 | 3.75 · | 5 wt % | 0.0617 | — | — |
| | | 10⁻⁴ | | | | |

### Procedure

The MAylO-L-La-Bn and methyl methacrylate monomers were columned through neutral alumina and initially charged in a Schlenk tube. Subsequently, the initiator dissolved in benzene was added, and the tube was closed with a septum. This solution was subjected to three freeze-pump procedures. The Schlenk tube, in the case of DMPA as initiator, was positioned in front of a UV lamp for 14 h; in the case of AlBN as initiator, it was heated to 70° C. for 16 h. The polymers were precipitated twice in ice-cold petroleum ether and dried on the Schlenk apparatus.

### Characterization

Appearance: colorless solids

### Yield

poly(MAylO-L-La-Bn-co-MMA) 1:1 (DMPA): 0.951 g, 85%, Mₙ=3130 g/mol, Ð=1.95

poly(MAylO-L-La-Bn-co-MMA) 1:2 (DMPA): 0.455 g, 84%, Mₙ=3130 g/mol, Ð=1.83

poly(MAylO-L-La-Bn-co-MMA) 2:1 (DMPA): 0.323 g, 90%, Mₙ=2750 g/mol, Ð=1.63

poly(MAylO-L-La-Bn-co-MMA) 1:1 (AlBN): 1.523 g, 99%, Mₙ=14480 g/mol, Ð=2.42

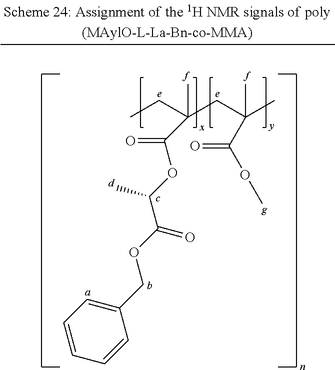

¹H NMR: (400 MHz; DMSO-d₆): δ[ppm]=0.66-1.20 (6H, Hf), 1.32-1.48 (3H, Hd), 1.62-2.08 (4H, He), 3.43-3.62 (3H, Hg), 4.85-5.07 (1H, Hc), 5.07-5.24 (2H, Hb), 7.28-7.41 (5H, Ha)

### EXAMPLE 13

### Poly(MAylO-D,L-La-Bn-co-MMA)

### Reaction Equation

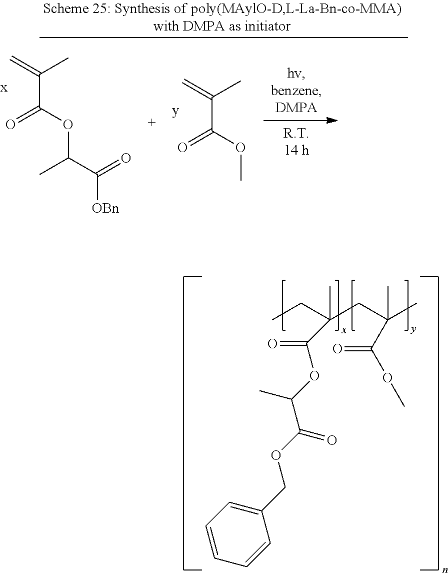

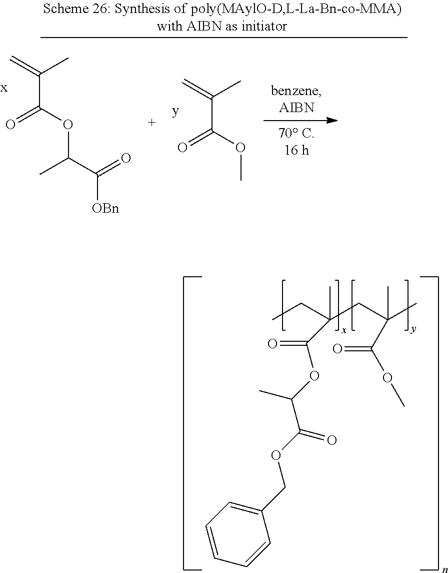

### Reaction

TABLE 22
Reaction table for the copolymerization of
MAylO-D,L-La-Bn with MMA in a ratio of 1:1

| Substance | M/(g/mol) | n/(mol) | Eq. | m/(g) | ρ/(g/mL) | V/(mL) |
|---|---|---|---|---|---|---|
| MAylO- | 248.28 | 0.0032 | 1.00 | 0.800 | — | — |
| D,L-La- | | | | | | |
| Bn | | | | | | |
| MMA | 100.12 | 0.0032 | 1.00 | 0.322 | 0.94 | 0.34 |
| Benzene | — | — | — | 5.63 | 0.88 | 6.40 |
| DMPA | 256.29 | 0.00022 | 5% wt | 0.0566 | — | — |

TABLE 23
Reaction table for the copolymerization of
MAylO-D,L-La-Bn with MMA in a ratio of 1:2

| Substance | M/(g/mol) | n/(mol) | Eq. | m/(g) | ρ/(g/mL) | V/(mL) |
|---|---|---|---|---|---|---|
| MAylO- | 248.28 | 0.0012 | 1.00 | 0.300 | — | — |
| D,L-La- | | | | | | |
| Bn | | | | | | |
| MMA | 100.12 | 0.0024 | 2.00 | 0.242 | 0.94 | 0.26 |
| Benzene | — | — | — | 3.17 | 0.88 | 3.60 |
| DMPA | 256.29 | 0.00011 | 5% wt | 0.0274 | — | — |

TABLE 24
Reaction table for the copolymerization of
MAylO-D,L-La-Bn with MMA in a ratio of 2:1

| Substance | M/(g/mol) | n/(mol) | Eq. | m/(g) | ρ/(g/mL) | V/(mL) |
|---|---|---|---|---|---|---|
| MAylO- | 248.28 | 0.0012 | 2.00 | 0.300 | — | — |
| D,L-La- | | | | | | |
| Bn | | | | | | |
| MMA | 100.12 | 0.0006 | 1.00 | 0.060 | 0.94 | 0.06 |
| Benzene | 78.11 | — | — | 1.58 | 0.88 | 1.80 |
| DMPA | 256.29 | 0.00007 | 5% wt | 0.0180 | — | — |

TABLE 25
Reaction table for the copolymerization of MAylO-D,L-La-Bn
with MMA in a ratio of 1:1 with AlBN as initiator

| Substance | M/(g/mol) | n/(mol) | Eq. | m/(g) | ρ/(g/mL) | V/(mL) |
|---|---|---|---|---|---|---|
| MAylO- | 248.28 | 4.43 · | 1.00 | 1.100 | — | — |
| D,L-La- | | 10⁻³ | | | | |
| Bn | | | | | | |
| MMA | 100.12 | 4.43 · | 1.00 | 0.4435 | 0.94 | 0.47 |
| | | 10⁻³ | | | | |
| Benzene | 78.11 | 0.039 | — | 3.1152 | 0.88 | 3.54 |
| AlBN | 164.21 | 3.75 · | 5 wt % | 0.0617 | — | — |
| | | 10⁻⁴ | | | | |

### Procedure

The procedure was analogous to the synthesis of the MAylO-L-La-Bn-co-MMA polymer.

### Characterization

Appearance: colorless solid

### Yield

poly(MAylO-D,L-La-Bn-co-MMA) 1:1 (DMPA): 0.809 g, 72%, Mₙ=3200 g/mol, Ð=1.78

poly(MAylO-D,L-La-Bn-co-MMA) 1:2 (DMPA): 0.434 g, 81%, Mₙ=2830 g/mol, Ð=1.69

poly(MAylO-D,L-La-Bn-co-MMA) 2:1 (DMPA): 0.333 g, 93%, Mₙ=2330 g/mol, Ð=1.74

poly(MAylO-D,L-La-Bn-co-MMA) 1:1 (AlBN): 1.533 g, 99%, Mₙ=14 990 g/mol, Ð=2.46

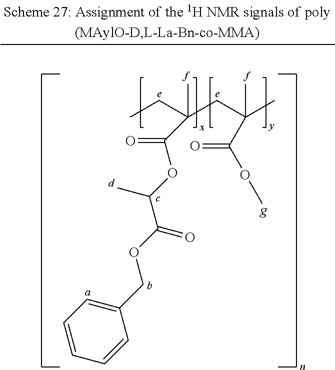

¹H NMR: (400 MHz; DMSO-d₆): δ[ppm]=0.64-1.07 (6H, Hf), 1.30-1.49 (3H, Hd), 1.62-2.08 (4H, He), 3.42-3.66 (3H, Hg), 4.85-5.06 (1H, Hc), 5.06-5.25 (2H, Hb), 7.23-7.46 (5H, Ha)

### EXAMPLE 14

### Poly(MAylO-Gly-Bn-co-MMA)

### Reaction Equations

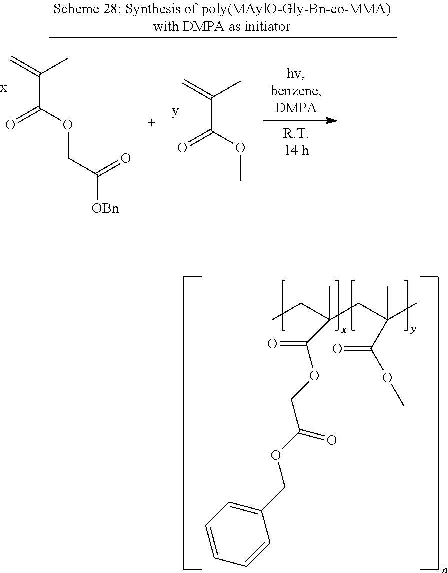

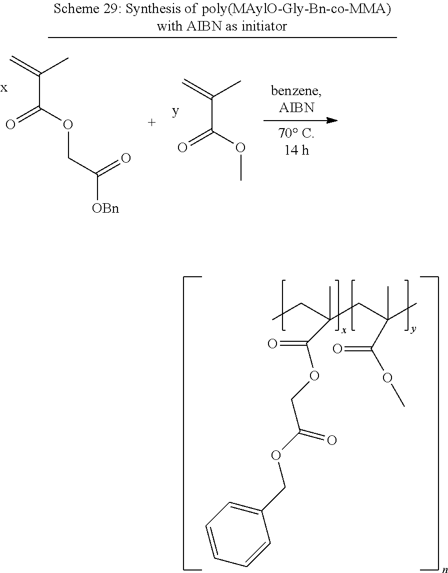

### Reaction

TABLE 26
Reaction table for the copolymerization of
MAylO-Gly-Bn with MMA in a ratio of 1:1

| Substance | M/(g/mol) | n/(mol) | Eq. | m/(g) | ρ/(g/mL) | V/(mL) |
|---|---|---|---|---|---|---|
| MAylO- | 234.25 | 0.0034 | 1.00 | 0.800 | — | — |
| Gly-Bn | | | | | | |
| MMA | 100.12 | 0.0034 | 1.00 | 0.342 | 0.94 | 0.36 |
| Benzene | — | — | — | 5.98 | 0.88 | 6.80 |
| DMPA | 256.29 | 0.00022 | 5% wt | 0.0575 | — | — |

TABLE 27
Reaction table for the copolymerization of
MAylO-Gly-Bn with MMA in a ratio of 1:2

| Substance | M/(g/mol) | n/(mol) | Eq. | m/(g) | ρ/(g/mL) | V/(mL) |
|---|---|---|---|---|---|---|
| MAylO- | 234.25 | 0.00128 | 1 | 0.300 | — | — |
| Gly-Bn | | | | | | |
| MMA | 100.12 | 0.00256 | 2 | 0.256 | 0.94 | 0.272 |
| Benzene | 78.11 | 0.0216 | — | 3.379 | 0.88 | 3.84 |
| DMPA | 256.29 | 0.0001 | 5% wt | 0.0278 | — | — |

TABLE 28
Reaction table for the copolymerization of MAylO-Gly-Bn
with MMA in a ratio of 2:1 with AlBN as initiator

| Substance | M/(g/mol) | n/(mol) | Eq. | m/(g) | ρ/(g/mL) | V/(mL) |
|---|---|---|---|---|---|---|
| MAylO- | 234.25 | 0.00128 | 2 | 0.300 | — | — |
| Gly-Bn | | | | | | |
| MMA | 100.12 | 0.00064 | 1 | 0.064 | 0.94 | 0.068 |
| Benzene | 78.11 | 0.0216 | — | 1.690 | 0.88 | 1.92 |
| DMPA | 256.29 | 0.00007 | 5% wt | 0.0182 | — | — |

TABLE 29
Reaction table for the copolymerization of
MAylO-Gly-Bn with MMA in a ratio of 1:1

| Substance | M/(g/mol) | n/(mol) | Eq. | m/(g) | ρ/(g/mL) | V/(mL) |
|---|---|---|---|---|---|---|
| MAylO- | 234.25 | 4.69 · | 1.00 | 1.100 | — | — |
| Gly-Bn | | 10⁻³ | | | | |
| MMA | 100.12 | 4.69 · | 1.00 | 0.4695 | 0.94 | 0.50 |
| | | 10⁻³ | | | | |
| Benzene | 78.11 | 0.042 | — | 3.300 | 0.88 | — |
| AlBN | 164.21 | 3.82 · | 5% wt | — | — | — |
| | | 10⁻⁴ | | | | |

### Procedure

The procedure was analogous to the synthesis of the MAylO-L-La-Bn-co-MMA polymer.

### Characterization

Appearance: colorless solid

### Yield

poly(MAylO-Gly-Bn-co-MMA) 1:1 (DMPA): 0.896 g, 79%, Mₙ=2790 g/mol, Ð=1.87

poly(MAylO-Gly-Bn-co-MMA) 1:2 (DMPA): 0.391 g, 70%, Mₙ=2680 g/mol, Ð=1.66

poly(MAylO-Gly-Bn-co-MMA) 2:1 (DMPA): 0.336 g, 92%, Mₙ=2760 g/mol, Ð=1.77

poly(MAylO-Gly-Bn-co-MMA) 1:1 (AlBN): 1.460 g, 93%, Mₙ=9800 g/mol, Ð=2.63

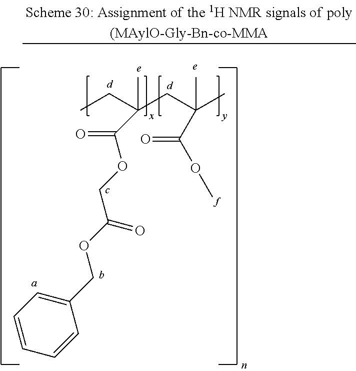

¹H NMR: (400 MHz; DMSO-d₆): δ[ppm]=0.61-1.26 (6H, He), 1.44-2.27 (4H, Hd), 3.43-3.68 (3H, Hf), 4.55-4.82 (2H, Hc), 5.09-5.26 (2H, Hb), 7.24-7.44 (5H, Ha)

### EXAMPLE 15

### Poly(MAylO-L-La-Bn-co-EA)

### Reaction Equation

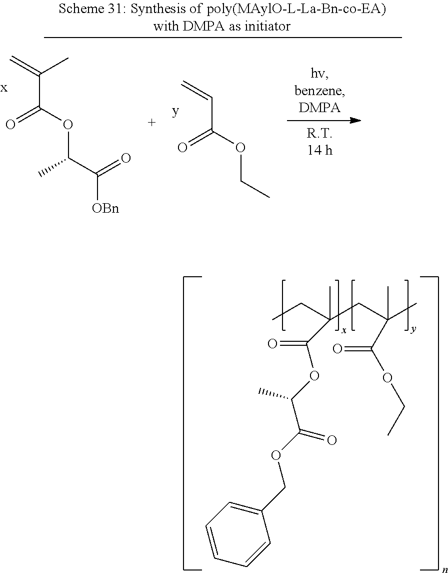

TABLE 30
Reaction table for the copolymerization of
MAylO-L-La-Bn with EA in a ratio of 1:1

| Substance | M/(g/mol) | n/(mol) | Eq. | m/(g) | ρ/(g/mL) | V/(mL) |
|---|---|---|---|---|---|---|
| MAylO-L- | 248.28 | 0.0048 | 1.00 | 1.20 | — | — |
| La-Bn | | | | | | |
| Ethyl | 100.12 | 0.0048 | 1.00 | 0.48 | 0.92 | 0.53 |
| acrylate | | | | | | |
| Benzene | 78.11 | 0.1082 | — | 8.45 | 0.88 | 9.60 |
| DMPA | 256.29 | 0.00033 | 5% wt | 0.0840 | — | — |

### Procedure

The MAylO-L-La-Bn and ethyl acrylate monomers were columned through neutral alumina and initially charged in a Schlenk tube. Subsequently, the initiator dissolved in benzene was added, and the tube was closed with a septum. This solution was subjected to three freeze-pump procedures and then positioned in front of a UV lamp for 14 h. The polymers were precipitated three times in ice-cold petroleum ether and dried on the Schlenk apparatus.

### Characterization

Appearance: colorless solid

### Yield

poly(MAylO-L-La-Bn-co-EA): 1.448 g, 86%, Mₙ=7830 g/mol, Ð=2.35

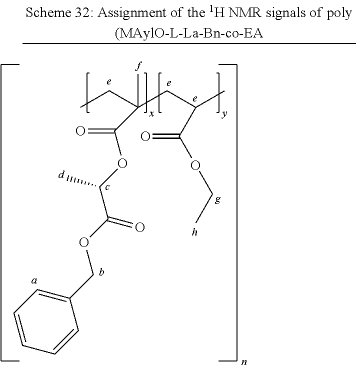

¹H NMR: (400 MHz; DMSO-d₆): δ[ppm]=0.78-1.02 (3H, Hf), 1.02-1.22 (3H, Hh), 1.28-1.45 (3H, Hd), 1.44-2.31 (5H, He), 3.80-4.12 (2H, Hg), 4.78-5.02 (1H, Hc), 5.02-5.23 (2H, Hb), 7.20-7.43 (5H, Ha)

### EXAMPLE 16

### Poly(MAylO-D,L-La-Bn-co-EA)

### Reaction Equation

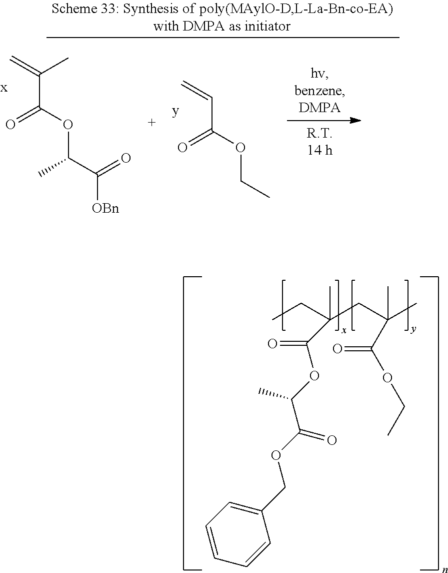

### Reaction

TABLE 31
Reaction table for the copolymerization of
MAylO-D,L-La-Bn with EA in a ratio of 1:1

| Substance | M/(g/mol) | n/(mol) | Eq. | m/(g) | ρ/(g/mL) | V/(mL) |
|---|---|---|---|---|---|---|
| MAylO- | 248.28 | 0.0048 | 1.00 | 1.20 | — | — |
| D,L-La- | | | | | | |
| Bn | | | | | | |
| Ethyl | 100.12 | 0.0048 | 1.00 | 0.48 | 0.92 | 0.53 |
| acrylate | | | | | | |
| Benzene | 78.11 | 0.1082 | — | 8.45 | 0.88 | 9.60 |
| DMPA | 256.29 | 0.00033 | 5% wt | 0.0840 | — | — |

### Procedure

The procedure was analogous to the synthesis of the MAylO-L-La-Bn-co-EA polymer.

### Characterization

Appearance: colorless solid

### Yield

poly(MAylO-D,L-La-Bn-co-EA): 1.234 g, 74%, Mₙ=5930 g/mol, Ð=2.15

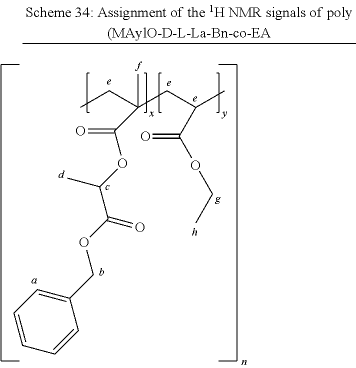

¹HNMR: (400 MHz; DMSO-d₆): δ[ppm]=0.79-1.00 (3H, Hf), 1.00-1.20 (3H, Hh), 1.25-1.46 (3H, Hd), 1.46-2.35 (5H, He), 3.76-4.11 (2H, Hg), 4.78-5.01 (1H, Hc), 5.01-5.20 (2H, Hb), 7.18-7.42 (5H, Ha)

### EXAMPLE 17

### Poly(MAylO-Gly-Bn-co-EA)

### Reaction Equation

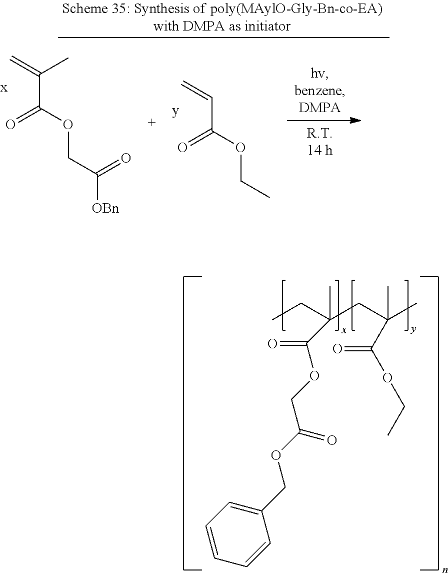

### Reaction

TABLE 32
Reaction table for the copolymerization
of MAylO-Gly-Bn with EA in a ratio of 1:1

| Substance | M/(g/mol) | n/(mol) | Eq. | m/(g) | ρ/(g/mL) | V/(mL) |
|---|---|---|---|---|---|---|
| MAylO- | 234.25 | 0.0047 | 1.00 | 1.10 | — | — |
| Gly-Bn | | | | | | |
| Ethyl | 100.12 | 0.0047 | 1.00 | 0.47 | 0.92 | 0.51 |
| acrylate | | | | | | |
| Benzene | 78.11 | 0.1059 | — | 8.27 | 0.88 | 9.4 |
| DMPA | 256.29 | 0.00031 | 5% wt | 0.0785 | — | — |

### Procedure

The procedure was analogous to the synthesis of the MAylO-L-La-Bn-co-EA polymer.

### Characterization

Appearance: colorless solid

### Yield

poly(MAylO-Gly-Bn-co-EA): 1.197 g, 76%, Mₙ=4680 g/mol, Ð=2.28

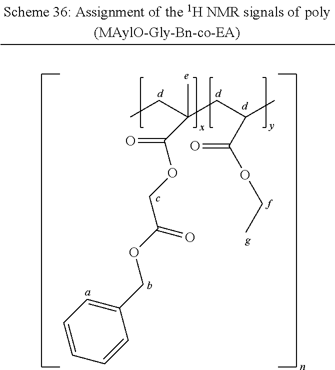

¹H NMR: (400 MHz; DMSO-d₆): δ[ppm]=0.73-1.03 (3H, He), 1.03-1.21 (3H, Hg), 1.24-2.38 (5H, Hd), 3.79-4.14 (2H, Hf), 4.48-4.82 (2H, Hc), 5.03-5.23 (2H, Hb), 7.21-7.45 (5H, Ha)

### EXAMPLE 18

### Poly(MA-co-MMA)

### Reaction equation

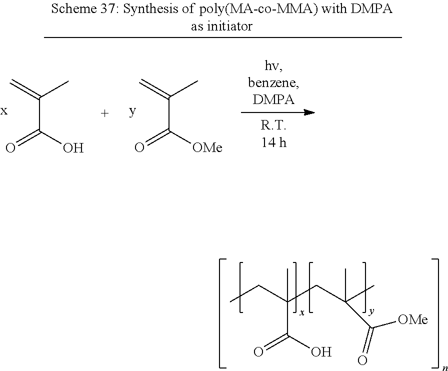

TABLE 33
Reaction table for the copolymerization of
methacrylic acid with methyl methacrylate

| Substance | M/(g/mol) | n/(mol) | Eq. | m/(g) | ρ/(g/mL) | V/(mL) |
|---|---|---|---|---|---|---|
| Meth- | 86.09 | 0.0174 | 1.00 | 1.50 | 1.02 | 1.47 |
| acrylic | | | | | | |
| acid | | | | | | |
| Methyl | 100.12 | 0.0174 | 1.00 | 1.74 | 0.92 | 1.89 |
| meth- | | | | | | |
| acrylate | | | | | | |
| Benzene | 78.11 | 0.3921 | — | 30.62 | 0.88 | 34.8 |
| DMPA | 256.29 | 0.00063 | 5% wt | 0.162 | — | — |

### Procedure

The methacrylic acid and methyl methacrylate monomers were columned through neutral alumina. Subsequently, they were initially charged in a Schlenk tube, and the initiator dissolved in benzene was added. The Schlenk tube was closed with a septum. This solution was subjected to three freeze-pump procedures and then positioned in front of a UV lamp for 14 h. The polymers were precipitated twice in ice-cold petroleum ether and dried on the Schlenk apparatus.

### Characterization

Appearance: colorless solid

### Yield

poly(MA-co-MMA): 3.166 g, 98%, analysis by means of DMF-GPC was not possible since polymer was undetectable on the column used.

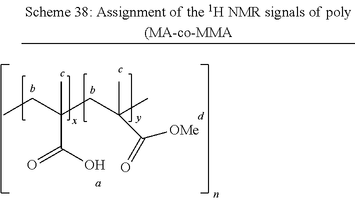

¹H NMR: (400 MHz; DMSO-d₆): δ[ppm]=0.61-1.21 (6H, Hc), 1.60-2.03 (4H, Hb), 3.48-3.59 (3H, Hd), 12.29-12.55 (1H, Ha)

### EXAMPLE 19

### Poly(MA-co-EA)

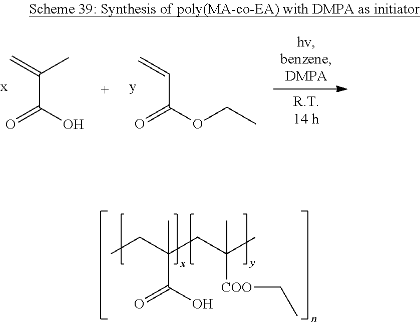

### Reaction

TABLE 34
Reaction table for the copolymerizations
of methacrylic acid with ethyl acrylate

| Substance | M/(g/mol) | n/(mol) | Eq. | m/(g) | ρ/(g/mL) | V/(mL) |
|---|---|---|---|---|---|---|
| Meth- | 86.09 | 0.0174 | 1.00 | 1.50 | 1.02 | 1.47 |
| acrylic | | | | | | |
| acid | | | | | | |
| Ethyl | 100.12 | 0.0174 | 1.00 | 1.74 | 0.94 | 1.85 |
| acrylate | | | | | | |
| Benzene | 78.11 | 0.3921 | — | 30.62 | 0.88 | 34.8 |
| DMPA | 256.29 | 0.00063 | 5% wt | 0.162 | — | — |

### Procedure

The procedure was analogous to the synthesis of the MA-co-MMA polymer.

### Characterization

Appearance: colorless solid

### Yield

poly(MA-co-EA): 3.122 g, 96%, analysis by means of GPC was not possible

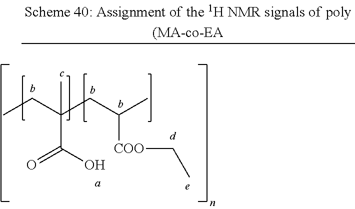

¹H NMR: (400 MHz; DMSO-d₆): δ[ppm]=0.79-1.08 (3H, Hc), 1.10-1.24 (3H, He), 1.30-2.39 (5H, Hb), 3.84-4.16 (2H, Hd), 12.25-12.48 (1H, Ha)

### EXAMPLE 20

### RAFT Polymerization of Poly(MAylO-Gly-Bn-co-MMA)

### Reaction Equation

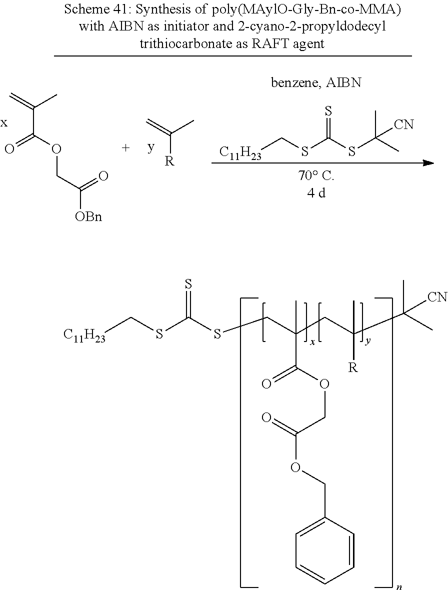

### Reaction

TABLE 35
Reaction table for the RAFT polymerization of MAylO-Gly-Bn with
methyl methacrylate to attain a molecular weight of 10 kg/mol

| Substance | M/(g/mol) | n/(mol) | Eq. | m/(g) | ρ/(g/mL) | V/(mL) |
|---|---|---|---|---|---|---|
| MAylO- | 234.25 | 0.00085 | 1.00 | 0.2000 | — | — |
| Gly-Bn | | | | | | |
| MMA | 100.12 | 0.00085 | 1.00 | 0.0851 | 0.94 | 0.09 |
| Benzene | 78.11 | 0.01960 | — | 1.5312 | 0.88 | 1.74 |
| AlBN | 164.21 | 1.4 · 10 ⁻⁶ | — | 0.0002 | — | — |
| 2-Cyano- | 345.63 | 2.8 · 10 ⁻⁵ | — | 0.0095 | — | — |
| 2-propyl- | | | | | | |
| dodecyl | | | | | | |
| trithio- | | | | | | |
| carbonate | | | | | | |

TABLE 36
Reaction table for the RAFT polymerization of MAylO-Gly-Bn with
methyl methacrylate to attain a molecular weight of 20 kg/mol

| Substance | M/(g/mol) | n/(mol) | Eq. | m/(g) | ρ/(g/mL) | V/(mL) |
|---|---|---|---|---|---|---|
| MAylO- | 234.25 | 0.00085 | 1.00 | 0.2000 | — | — |
| Gly-Bn | | | | | | |
| MMA | 100.12 | 0.00085 | 1.00 | 0.0851 | 0.94 | 0.09 |
| Benzene | 78.11 | 0.01938 | — | 1.5136 | 0.88 | 1.72 |
| AlBN | 164.21 | 7.3 · 10⁻⁷ | — | 0.0001 | — | — |
| 2-Cyano- | 345.63 | 1.4 · 10⁻⁵ | — | 0.0049 | — | — |
| 2-propyl- | | | | | | |
| dodecyl | | | | | | |
| trithio- | | | | | | |
| carbonate | | | | | | |

### Procedure

The methyl methacrylate and MAylO-Gly-Bn monomers were columned through neutral alumina. Subsequently, they were initially charged in a Schlenk tube. The AlBN initiator and the 2-cyano-2-propyldodecyl trithiocarbonate RAFT agent were dissolved in benzene and transferred into the Schlenk tube. The Schlenk tube was closed with a glass stopper and the solution was subjected to three freeze-pump procedures. The solution was heated to 70° C. for 4 days. The yellow solution was precipitated twice in ice-cold petroleum ether, and the polymer was dried on the Schlenk apparatus.

### Characterization

10 kg/mol: 0.242 g, 86%, Mₙ=5810 g/mol, Ð=1.33, yellowish solid

20 kg/mol: 0.275 g, 97% Mₙ=10 730 g/mol, Ð=1.42, yellowish solid

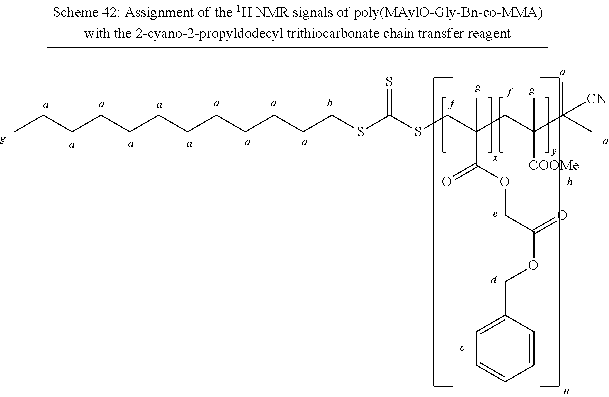

### Assignment of the ¹H NMR Signals

10 kg/mol:

¹H NMR: (400 MHz; DMSO-d₆): δ[ppm]=0.68-1.09 (172H, Hg), 1.13-1.33 (36H, Ha), 1.33-2.11 (104H, Hf), 3.23-3.29 (2H, Hb), 3.42-3.63 (81H, Hh), 4.56-4.79 (69H, He), 5.08-5.24 (72H, Hd), 7.25-7.43 (180H, Hc)

20 kg/mol:

¹H NMR: (400 MHz; DMSO-d₆): δ[ppm]=0.68-1.09 (445H, Hg), 1.13-1.33 (26H, Ha), 1.33-2.11 (260H, Hf), 3.23-3.29 (2H, Hb), 3.42-3.63 (225H, Hh), 4.56-4.79 (152H, He), 5.08-5.24 (161H, Hd), 7.25-7.43 (412H, Hc)

### EXAMPLE 21

### RAFT Polymerization of Poly(MAylO-L-La-Bn-co-MMA)

### Reaction Equation

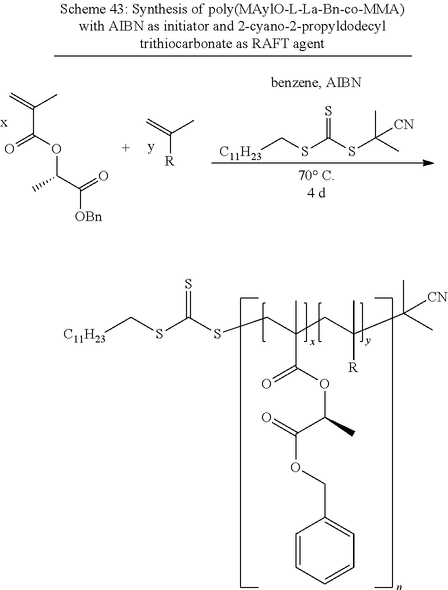

### Reaction

TABLE 37
Reaction table for the RAFT polymerization of
poly(MAylO-D,L-La-Bn) with methyl methacrylate
to attain a molecular weight of 10 kg/mol

| Substance | M/(g/mol) | n/(mol) | Eq. | m/(g) | ρ/(g/mL) | V/(mL) |
|---|---|---|---|---|---|---|
| MAylO- | 248.28 | 0.0008 | 1.00 | 0.2000 | — | — |
| L-La-Bn | | | | | | |
| MMA | 100.12 | 0.0008 | 1.00 | 0.0807 | 0.94 | 0.09 |
| Benzene | 78.11 | 0.0184 | — | 1.4432 | 0.88 | 1.64 |
| AlBN | 164.21 | 1.4 · 10⁻⁶ | — | 0.0002 | — | — |
| 2-Cyano- | 345.63 | 2.8 · 10⁻⁵ | — | 0.0097 | — | — |
| 2-propyl- | | | | | | |
| dodecyl | | | | | | |
| trithio- | | | | | | |
| carbonate | | | | | | |

TABLE 38
Reaction table for the RAFT polymerization of MAylO-L-La-Bn with
methyl methacrylate to attain a molecular weight of 20 kg/mol

| Substance | M/(g/mol) | n/(mol) | Eq. | m/(g) | ρ/(g/mL) | V/(mL) |
|---|---|---|---|---|---|---|
| MAylO-L- | 248.28 | 0.0008 | 1.00 | 0.2000 | — | — |
| La-Bn | | | | | | |
| MMA | 100.12 | 0.0008 | 1.00 | 0.0807 | 0.94 | 0.09 |
| Benzene | 78.11 | 0.0184 | — | 1.4432 | 0.88 | 1.64 |
| AlBN | 164.21 | 7.3 · 10⁻⁷ | — | 0.0001 | — | — |
| 2-Cyano- | 345.63 | 1.4 · 10⁻⁵ | — | 0.0048 | — | — |
| 2-propyl- | | | | | | |
| dodecyl | | | | | | |
| trithio- | | | | | | |
| carbonate | | | | | | |

### Procedure

The procedure was analogous to the synthesis of the MAylO-Gly-Bn-co-MMA polymer by RAFT.

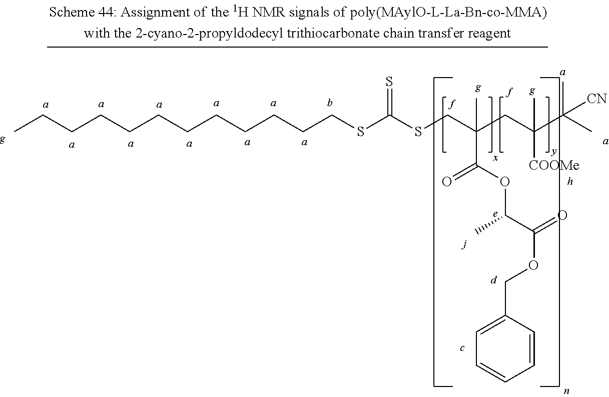

### Characterization

10 kg/mol: 0.182 g, 65%, Mₙ=4670 g/mol, Ð=1.29, yellowish solid

20 kg/mol: 0.264 g, 94%, Mₙ=10 930 g/mol, Ð=1.34, yellowish solid

### Assignment of the 1H NMR Signals

10 kg/mol:

¹HNMR: (400 MHz; DMSO-d₆): δ[ppm]=0.67-1.08 (126H, Hg), 1.17-1.27 (21H, Ha), 1.27-1.48 (91H, Hi), 1.52-2.21 (85H, Hf), 3.21-3.30 (2H, Hb), 3.42-3.66 (58H, Hh), 4.86-5.05 (27H, He), 5.05-5.24 (53H, Hd), 7.25-7.41 (134H, Hc)

20 kg/mol:

¹H NMR: (400 MHz; DMSO-d₆): δ[ppm]=0.67-1.08 (317H, Hg), 1.17-1.27 (23H, Ha), 1.27-1.48 (194H, Hi), 1.52-2.21 (195H, Hf), 3.21-3.30 (2H, Hb), 3.42-3.66 (162H, Hh), 4.86-5.05 (56H, He), 5.05-5.24 (107H, Hd), 7.25-7.41 (280H, Hc)

### EXAMPLE 22

### Modifications on the Polymer

### Reaction Equation

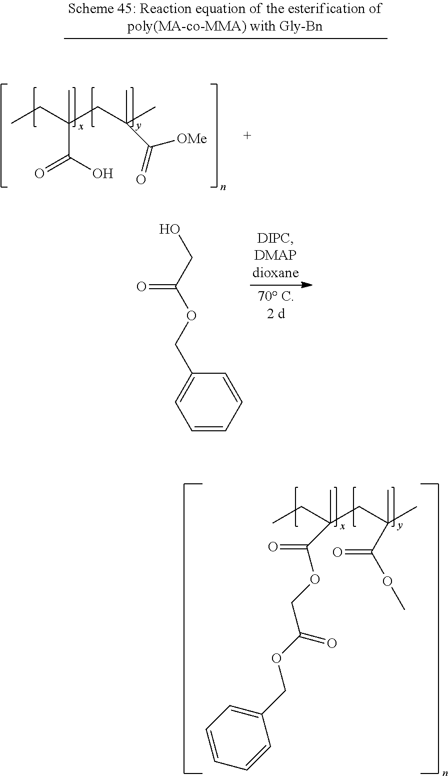

### Reaction

TABLE 39
Reaction table for the coupling of poly(MA-co-MMA) with Gly-Bn

| Substance | M/(g/mol) | n/(mol) | Eq. | m/(g) | ρ/(g/mL) | V/(mL) |
|---|---|---|---|---|---|---|
| poly(MA- | 186.21 | 5.37 · 10⁻⁴ | 1.0 | 0.1000 | — | — |
| co-MMA) | | | | | | |
| Gly-Bn | 166.17 | 8.05 · 10⁻⁴ | 1.5 | 0.1338 | — | — |
| DIPC | 126.20 | 8.05 · 10⁻⁴ | 1.5 | 0.1016 | 0.81 | 0.13 |
| DMAP | 122.17 | 5.37 · 10⁻⁵ | 0.1 | 0.0066 | — | — |
| Dioxane | 88.11 | 5.84 · 10⁻² | — | 5.1500 | 1.03 | 5.00 |

### Procedure

The MA-co-MMA polymer and DMAP were initially charged in a 100 mL round-bottom flask and dissolved in dioxane. Subsequently, DMAP and Gly-Bn were added. The flask was heated to 70° C. for two days. The resultant solution was precipitated twice in ice-cold petroleum ether and dried on the Schlenk apparatus.

### Characterization

Appearance: colorless solid Yield:

### Yield

poly(MAylO-Gly-Bn-co-MMA): 0.165 g, 92%, Mₙ=5880 g/mol, Ð=3.83

The ¹HNMR spectrum and the assignment correspond to that of poly(MAylO-Gly-Bn-co-MMA) in Scheme 42.

### EXAMPLE 23

### Hydrogenation

### Reaction Equation

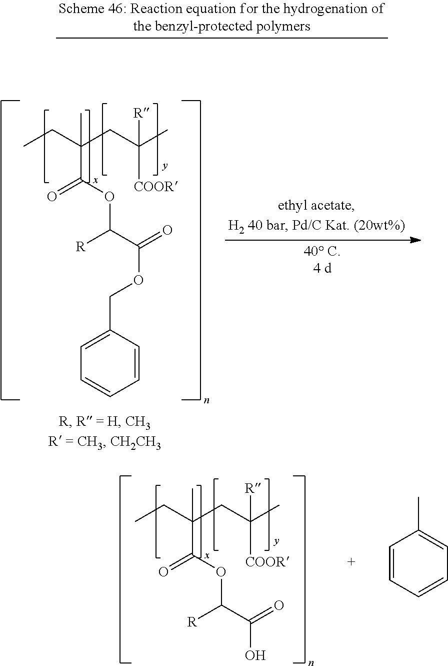

### Reaction

TABLE 40
Reaction table for the hydrogenations
of the benzyl-protected polymers

| Reaction | Substance | M/(g/mol) | n/(mol) | Eq. | m/(g) |
|---|---|---|---|---|---|
| 1 | poly(MAylO- | 348.40 | 2.7 · | 1 | 0.9510 |
| | L-La-Bn-co- | | 10⁻³ | | |
| | MMA) (DMPA) | | | | |
| | Pd/C | 166.42/12.01 | — | 20 wt % | 0.2051 |
| 2 | poly(MAylO- | 348.40 | 2.3 · | 1 | 0.8090 |
| | D,L-La-Bn-co- | | 10⁻³ | | |
| | MMA) (DMPA) | | | | |
| | Pd/C | 106.42/12.01 | — | 20 wt % | 0.2099 |
| 3 | poly(MAylO- | 334.37 | 2.7 · | 1 | 0.8960 |
| | Gly-Bn-co- | | 10⁻³ | | |
| | MMA) (DMPA) | | | | |
| | Pd/C | 106.42/12.01 | — | 20 wt % | 0.1911 |
| 4 | poly(MAylO- | 348.40 | 4.4 · | 1 | 1.5230 |
| | L-La-Bn-co- | | 10⁻³ | | |
| | MMA) (AlBN) | | | | |
| | Pd/C | 106.42/12.01 | — | 20 wt % | 0.3067 |
| 5 | poly(MAylO- | 348.40 | 4.4 · | 1 | 1.5330 |
| | D,L-La-Bn-co- | | 10⁻³ | | |
| | MMA) (AlBN) | | | | |
| | Pd/C | 106.42/12.01 | — | 20 wt % | 0.3094 |
| 6 | poly(MAylO- | 334.37 | 4.4 · | 1 | 1.4600 |
| | Gly-Bn-co- | | 10⁻³ | | |
| | MMA) (AlBN) | | | | |
| | Pd/C | 106.42/12.01 | — | 20 wt % | 0.3077 |
| 7 | poly(MAylO- | 348.40 | 4.2 · | 1 | 1.4480 |
| | L-La-Bn-co- | | 10⁻³ | | |
| | EA) (DMPA) | | | | |
| | Pd/C | 106.42/12.01 | — | 20 wt % | 0.2916 |
| 8 | poly(MAylO- | 348.40 | 3.5 · | 1 | 1.2340 |
| | D,L-La-Bn-co- | | 10⁻³ | | |
| | EA) (DMPA) | | | | |
| | Pd/C | 106.42/12.01 | — | 20 wt % | 0.2468 |
| 9 | poly(MAylO- | 334.37 | 3.6 · | 1 | 1.1970 |
| | Gly-Bn-co- | | 10⁻³ | | |
| | EA) (DMPA) | | | | |
| | Pd/C | 106.42/12.01 | — | 20 wt % | 0.2394 |

The molecular weight of the polymers is based on the repeat unit; the palladium/carbon catalyst has a palladium content of 5 wt %.

### Procedure

The polymer was dissolved in 100 mL of ethyl acetate and transferred together with the Pd/C catalyst into a pressure reactor. The latter was closed, and H₂ gas was introduced up to a pressure of 40 bar. The solution was heated to 40° C. while stirring for 4 days. The reactor was then opened cautiously, and the black liquid was filtered through a Celite column. The colorless liquid was concentrated under reduced pressure and dried on the Schlenk apparatus. This gives colorless, relatively porous solids.

Characterization

Appearance: colorless solids

Yields: quantitative

In addition, the invention is elucidated in detail by figures. The specific figures show:

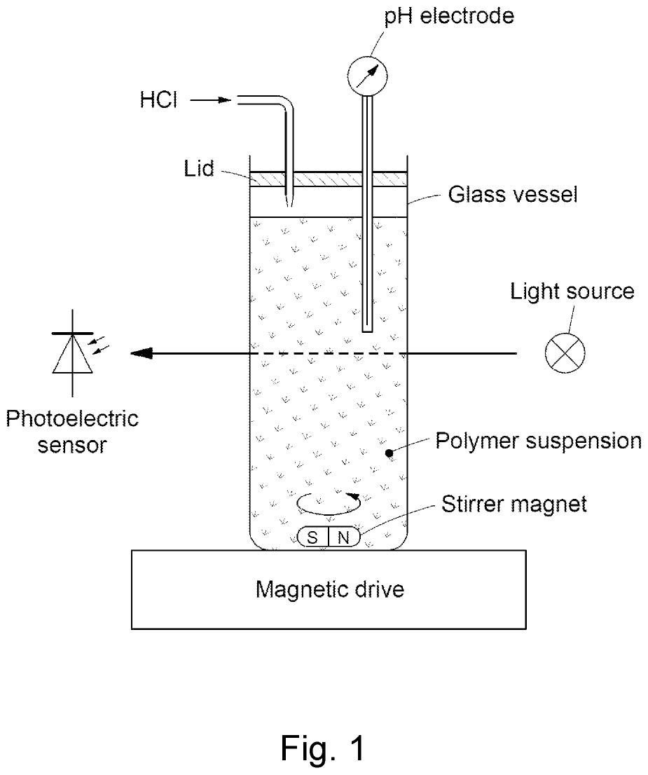

FIG. 1 an apparatus for determination of the solubility of polymers as a function of pH;

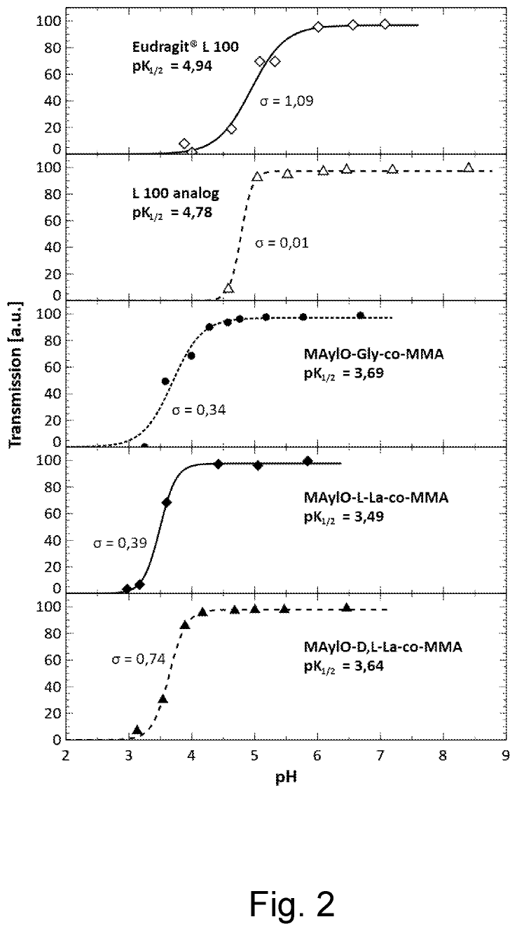

FIG. 2 the result of solubility measurements on Eudragit® L 100 and analogous polymers of the invention in diagram form;

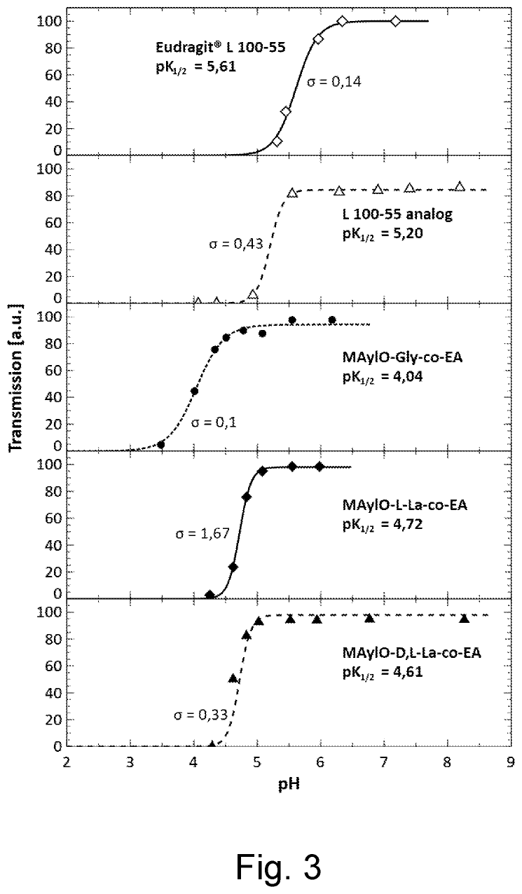

FIG. 3 the result of solubility measurements on Eudragit® L 100-55 and analogous polymers of the invention in diagram form;

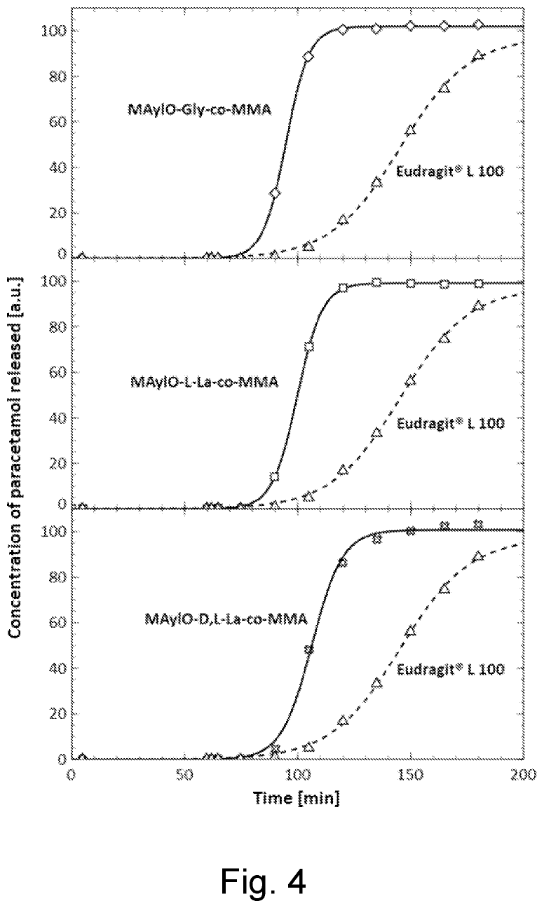

FIG. 4 the release of paracetamol from capsules coated with Eudragit® L 100 and analogous polymers;

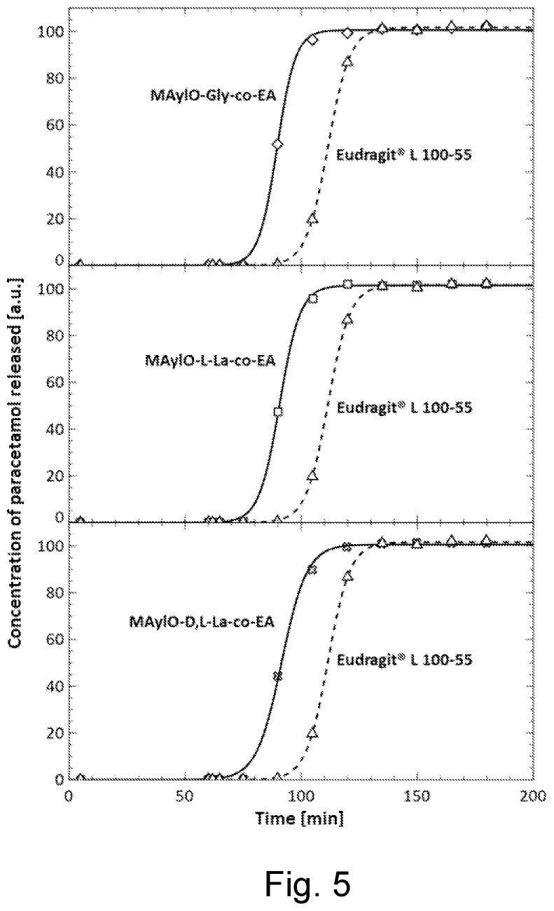

FIG. 5 the release of paracetamol from capsules coated with Eudragit® L 100-55 and analogous polymers.

EXAMPLE 24

### Solubility

FIGS. 2 and 3 show the measurement results for solubility of polymers of the Eudragit® L 100 and Eudragit® L 100-55 type by comparison with polymers of the invention in the form of diagrams. As well as the measurements represented as dots, diagrams show fitted curves that are each based on a pH-dependent function of the following type:

$$f ( p H ) = a \cdot [ 2 + tanh ( \frac{p H - p {K}_{1 / 2}}{c} ) ]$$

with the parameters of a, pK_{1/2} and c to be fitted. pK_{1/2} corresponds here to the pH at which about 50% of the respective polymer is solvated. The diagrams further state a standard error o for each fitted curve, which is calculated as the square root of the mean square between the fitted curve and the measurements, according to the following relationship:

$$σ = \sqrt{\frac{1}{2} {\sum }_{i = 1}^{n} {[ T ( p {H}_{i} ) - f ( p {H}_{i} ) ]}^{2} }$$

in which T(pHᵢ) denotes the transmission measured at pH, and n the number of measurements.

It is apparent from the measurement results for solubility that are reproduced in FIGS. 2 and 3 that the polymers of the invention are solvated at lower pH compared to Eudragit® polymers of the L 100 and L 100-55 type. For instance, at a pH of 4.5, the light transmittance of a Eudragit® L 100 suspension is less than 20%, compared to 90% to 100% for inventive polymers of the MAylO-Gly-co-MMA, MAylO-L-La-co-MMA and MAylO-D,L-La-co-MMA type. The situation is similar for Eudragit® L 100-55 and inventive polymers of the MAylO-Gly-co-EA, MAylO-L-La-co-EA and MAylO-D,L-La-co-EA type.

For comparative purposes, the inventors also synthesized, by means of free-radical polymerization, polymers analogous to Eudragit® L 100 and Eudragit® L 100-55, referred to as “L 100 analog” and “L 100-55 analog” respectively, and examined the solubility thereof. The polymers of the “L 100 analog” and “L 100-55 analog” type dissolve at slightly lower pH than the Eudragit® polymers prepared by means of anionic polymerization. The dissolution characteristics of the “L 100 analog” and “L 100-55 analog” polymers are likely to be attributable to a lower molecular weight.

### EXAMPLE 25

### Release of Paracetamol

FIGS. 4 and 5 show measurement results for the release of the active ingredient paracetamol from coated capsules under physiological conditions, i.e. at a temperature of 37° C., pH 2, within a time interval of 0-60 min, and pH 6.5 for the time interval of >60 min. The diagrams show discrete measurements or measurement points and fitted curves. The fitted curves are based on a function of the same type as described above in example 24, using time rather than the pH as the independent variable.

It is apparent from FIGS. 4 and 5 that paracetamol is not released at a pH of 2 within the first 60 minutes for any of the capsule coatings tested. Moreover, the measurement results show that the active ingredient paracetamol is respectively released from the capsules coated with the inventive polymers about 60 minutes and about 25 minutes earlier compared to Eudragit® L 100 and Eudragit® L 100-55. The smaller time difference of about 25 minutes in the case of release from capsules coated with MAylO-Gly-co-EA, MAylO-L-La-co-EA and MAylO-D,L-La-co-EA compared to Eudragit® L 100-55 is attributable to the fact that the polymers in question dissolve at a pH in the range from 4.0 to 4.7 (cf. example 24). By contrast, the polymers of the MAylO-Gly-co-MMA, MAylO-L-La-co-MMA and MAylO-D,L-La-co-MMA type already dissolve at a pH in the range from 3.5 to 3.7.

### List of Abbreviations

The abbreviations used in the context of the present description have the meaning given below, with some of the abbreviations for copolymers between parentheses preceded by the word “poly”; for example, the abbreviations “MAylO-Gly-Bn-co-EA” and “poly(MAylO-Gly-Bn-co-EA)” refer to the same copolymer:
AlBN . . . azoisobutyronitrile
ATRP . . . atom transfer radical polymerization
Eq. . . . equivalents
AS . . . acrylic acid residue (—CH[(C═O)—]CH₂—)
Ayl . . . acryloyl group (CH₂═CH—(C═O)—)
AylO . . . acryloyloxy group (CH₂═CH—(C═O)—O—)
AylO-Gly-Bn . . . 2-acryloyloxyethanoate benzyl ester
AylO-L-La-Bn . . . (S)-2-acryloyloxypropionate benzyl ester
AylO-D,L-La-Bn . . . 2-acryloyloxypropionate benzyl ester
AylO-Gly-Bn-co-EA . . . 2-acryloyloxyethanoate benzyl-ethyl acrylate copolymer
AylO-L-La-Bn-co-EA . . . (S)-2-acryloyloxypropionate benzyl-ethyl acrylate copolymer
AylO-D,L-La-Bn-co-EA . . . 2-acryloyloxypropionate benzyl-ethyl acrylate copolymer
AylO-Gly-Bn-co-MMA . . . 2-acryloyloxyethanoate benzyl-methyl methacrylate copolymer
AylO-L-La-Bn-co-MMA . . . (S)-2-acryloyloxypropionate benzyl-methyl methacrylate copolymer
AylO-DfL-La-Bn-co-MMA 2-acryloyloxypropionate benzyl-methyl methacrylate copolymer
BHT . . . butylhydroxytoluene
CFRP . . . controlled free radical polymerization
DBU . . . 1,8-diazabicyclo[5.4.0]undec-7-ene
DIPC . . . diisopropylcarbodiimide
D,L-La-Bn . . . 2-hydroxypropionate benzyl ester
DMAP . . . 4-(N,N-dimethylamino)pyridine)
DMF . . . N,N-dimethylformamide
DMPA . . . 2,2-dimethoxy-2-phenylacetophenone
DMSO . . . dimethyl sulfoxide
EA . . . .ethyl acrylate residue (—CH[(C═O)OCH₂CH₃]CH₂—)
EMA . . . ethyl methacrylate residue (—C(CH₃)[(C═0)0CH₂CH₃]CH₂—)
EtAc . . . ethyl acetate
Gly-Bn . . . hydroxyethanoate benzyl ester
GPC . . . gel permeation chromatography
L-La-Bn . . . (S)-2-hydroxypropionate benzyl ester
MA . . . .methyl aerylate residue (—CH[(C═O)OCH₃]CH₂—)
MA-co-MMA . . . methacrylica cid-methyl methacrylate copolymer
MA-co-EA . . . methacrylica cid-ethyl acrylate copolymer
MAS . . . methacrylie acid residue (—C(CH₃)[(C═O)—]CH₂—)
MAyl . . . methacryloyl-group ( CH₂═C(CH₃)—(C═O)—)
MAylO . . . methacryloyloxy group ( CH₂═C(CH₃)—(C═O)—O—)
MAylO-Gly-Bn . . . 2-methacryloyloxyethanoate benzyl ester
MAylO-L-La-Bn . . . (S)-2-methacryloyloxypropionate benzyl ester
MAylO-D,L-La-Bn . . . 2-methacryloyloxypropionate benzyl ester
MAylO-Gly-Bn-co-EA . . . 2-methacryloyloxyethanoate benzyl-ethyl acrylate copolymer
MAylO-L-La-Bn-co-EA . . . (S)-2-methacryloyloxypropionate benzyl-ethyl acrylate copolymer
MAylO-D,L-La-Bn-co-EA . . . 2-methacryloyloxypropionate benzyl-ethyl acrylate copolymer
MAylO-Gly-Bn-co-MMA . . . 2-methacryloyloxyethanoate benzyl-methyl methacrylate copolymer
MAylO-L-La-Bn-co-MMA (S)-2-methacryloyloxypropionate benzyl-methyl methacrylate copolymer
MAylO-D,L-La-Bn-co-MMA . . . 2-methacryloyloxypropionate benzyl-methyl methacrylate copolymer
MMA . . . methyl methacrylate residue (—C(CH₃)[(C═O)OCH₃]CH₂—)
RAFT . . . reversible addition fragmentation chain transfer polymerization

In the context of the present invention, the term “radical polymerization” encompasses methods such as free-radical polymerization, controlled free radical polymerization (CFRP), reversible addition fragmentation chain transfer polymerization (RAFT) and atom transfer radical polymerization (ATRP).

The copolymers of the invention may be either random copolymers or block copolymers. Accordingly, the IUPAC-conformant term “-co-” in the polymer structural formulae of the present invention includes the IUPAC-conformant terms “-stat-” and “-block-”.

### Test Methods

In the context of the present invention, weights and weight distributions of the copolymers produced are determined by means of gel permeation chromatography (GPC or SEC) in dimethylformamide (DMF) at a temperature in the range from 25 to 30° C., standard pressure (985-1010 hPa) and typical humidity (40-100% rH) (source: measurement station of the Institute for Atmospheric Physics, Johannes Gutenberg University of Mainz).

All chemicals and solvents, unless stated otherwise, were sourced from commercial suppliers (Acros, Sigma-Aldrich, Fisher Scientific, Fluka, Riedel-de-Haën, Roth) and—apart from the drying of the solvents and monomers—used without further purification. Deuterated solvents were sourced from Deutero GmbH (Kastellaun, Germany).

### Gel Permeation Chromatography (GPC or SEC)

GPC or SEC measurements were conducted according to DIN 55672-3 2016-01 at a temperature of 25 to 30° C. on an Agilent 1100 HPLC system with refractive index detector (Agilent 2160 Infinity RI detector), UV detector (275 nm), online viscometer and an SDV column set (SDV 103, SDV 105, SDV 106) from Polymer Standard Service GmbH (referred to hereinafter as PSS). Dimethylformamide (DMF) was used as solvent for the polymers to be analyzed and as eluent at a volume flow rate of 1 mL·min⁻¹. The polymers to be analyzed, having been dissolved in DMF, were injected into the GPC column by means of a Waters 717 plus autosampler. Calibration was effected using polystyrene standards from PSS. The elugrams were evaluated with the aid of the PSS WinGPC Unity software from PSS.

### NMR Spectroscopy

¹H and ¹³C NMR spectra were recorded on an Avance II 400 (400 MHz, 5 mm BBFO head with z gradient and ATM) from Bruker, with a frequency of 400 MHz (¹H) or 101 MHz (¹³C). For kinetic in situ ¹H NMR measurements, a Bruker Avance III HD 400 spectrometer equipped with a 5 mm BBFO SmartProbe sensor (Z gradient probe), ATM and SampleXPress 60 autosampler was used. Chemical shifts are reported in ppm and are based on the proton signal of the deuterated solvent.

### Solubility and Active Ingredient Release

The solubility of inventive and known polymers of the Eudragit® class is determined by means of optical transmittance measurements at a temperature of 37° C. For this purpose, the polymer to be examined in each case is dissolved or suspended in a concentration of 5 mg/mL in a basic NaOH-buffered bath, and the pH is lowered stepwise by means of titration of 0.1 M HCl solution. As the pH is lowered, the polymer is protonated and precipitates out, which scatters and attenuates the light.

The apparatus used for the measurement of solubility is shown in schematic form in FIG. 1. The polymer solution or suspension is in a glass vessel closed with a lid and heated to 37° C. by means of Peltier elements. A magnetic stirrer is disposed in the glass vessel, i.e. in the polymer suspension, which is rotated by means of a magnetic drive. A light beam emitted by a light source passes through the walls of the glass vessel and the polymer suspension between them, and hits a photoelectric sensor, for example a photodiode, with which the intensity of the light beam transmitted is measured. The apparatus further comprises a reservoir vessel (not shown in FIG. 1) for HCl, which is connected via a conduit to the interior of the glass vessel. Disposed in the conduit is a metering or titration valve (not shown in FIG. 1) with which the amount of HCl supplied to the polymer suspension per unit time is controlled. The optical measurement of transmittance is conducted with the aid of a Jasco V-640 spectrophotometer.

In addition, paracetamol-containing capsules are coated with inventive polymers and Eudragit® L 100 and Eudragit® L 100-55, and the release of paracetamol is examined with simulation of the physiological conditions in the gastrointestinal tract. The apparatus used for the simulation—as obtainable, for example, from Erweka GmbH—corresponds to apparatus 1 in the European Pharmacopoeia. At given times, fixed, negligibly small amounts of liquid compared to the contents of the test vessel are withdrawn, and the paracetamol concentration is determined photometrically at a wavelength of 243 nm.

Multiple paracetamol-containing capsules of identical form are respectively coated with a coating of Eudragit® L 100 and Eudragit® L 100-55 and the inventive polymers of the MAylO-Gly-co-MMA, MAylO-L-La-co-MMA, MAylO-D,L-La-co-MMA, MAylO-Gly-co-EA, MAylO-L-La-co-EA and MAylO-D,L-La-co-EA type. In the course of a test series, the viscosity of the respective polymer solution is adjusted such that the weight per unit area of the coating or the increase in weight of the capsules as a result of the coating conforms to an accuracy of ±3 %.

3 to 4 capsules coated with one of the polymers to be examined in each case are introduced into 900 mL of a test solution having a pH of 2. Over a period of 60 minutes, the test solution containing the capsules is stirred while retaining the pH of 2 and a temperature of 37° C., in order to simulate the acidic environment of the stomach. Subsequently, the pH is raised to 6.5 by replacing the test solution with phosphate buffer.

1. A copolymer comprising the following structure:
poly(MAₓ-co-[AS—(O—R)ₙ—OH]_{y}-co-[AS—OH]_{z}),
poly(MMAₓ-co-[AS—(O—R)ₙ—OH]_{y}-co-[AS—OH]_{z}),
poly(EAₓ-co-[AS—(O—R)ₙ—OH]_{y}-co-[AS—OH]_{z}),
poly(EMAₓ-co-[AS—(O—R)ₙ—OH]_{y}-co-[AS—OH]_{z}),
poly(MAₓ-co-[AS—(O—R)ₙ—OH]_{y}-co-[MAS—OH]_{z}),
poly(MMAₓ-co-[AS—(O—R)ₙ—OH]_{y}-co-[MAS—OH]_{z}),
poly(EAₓ-co-[AS—(O—R)ₙ—OH]_{y}-co-[MAS—OH]_{z}),
poly(EMAₓ-co-[AS—(O—R)ₙ—OH]_{y}-co-[MAS—OH]_{z}),
poly(MAₓ-co-[MAS—(O—R)ₙ—OH]_{y}-co-[AS—OH]_{z}),
poly(MMAₓ-co-[MAS—(O—R)ₙ—OH]_{y}-co-[AS—OH]_{z}),
poly(EAₓ-co-[MAS—(O—R)ₙ—OH]_{y}-co-[AS—OH]_{z}),
poly(EMAₓ-co-[MAS—(O—R)ₙ—OH]_{y}-co-[AS—OH]_{z}),
poly(MAₓ-co-[MAS—(O—R)ₙ—OH]_{y}-co-[MAS—OH]_{z}),
poly(MMAₓ-co-[MAS—(O—R)ₙ—OH]_{y}-co-[MAS—OH]_{z}),
poly(EAₓ-co-[MAS—(O—R)ₙ—OH]_{y}-co-[MAS—OH]_{z}),
or
poly(EMAₓ-co-[MAS—(O—R)ₙ—OH]_{y}-co-[MAS—OH]_{z});
wherein MA=methyl acrylate residue (—CH[(C═O)OCH₃]CH₂—), MMA=methyl methacrylate residue (—C(CH₃)[(C═O)OCH₃]CH₂—), EA=ethyl acrylate residue (—CH[(C═O)OCH₂CH₃]CH₂—), EMA=ethyl methacrylate residue (—C(CH₃)[(C═O)OCH₂CH₃]CH₂—); AS=acrylic acid residue (—CH[(C═O)—]CH₂—), MAS=methacrylic acid residue (—C(CH₃)[(C═O)—]CH₂—); and
wherein R=—CH₂(C═O)—, R=—CH(CH₃)(C═O)—, R=—CH(CH₂CH₃)(C═O)—, R=—C(CH₃)₂(C═O)—, R=—C(CH₃)(COCH₃)(C═O)—, R=—CH(Ph)(C═O)—, R=—CH[(CH₂)₂SCH₃](C═O)—, R=—CH(CH₂COOH)(C═O)—, R=—CH(COOH)(C═O)—, R=—C(CH₂COOH)₂(C═O)—, R=—CH(COOH)CH(CH₂COOH)(C═O)— or R=—CH(COOH)(CHOH)(C═O)—; and
wherein n is an integer with 1≤n≤20 and x, y, z denote the relative molar proportions of the monomer units with 1≤x≤20, 1≤y≤20 and 0≤z≤20.

2. A copolymer as claimed in claim 1, wherein it has the structure:
poly(MAₓ-co-[AS—O—R—OH]_{y}-co-[AS—OH]_{z}),
poly(MAₓ-co-[MAS—O—R—OH]_{y}-co-[MAS—OH]_{z}),
poly(MMAₓ-co-[AS—O—R—OH]_{y}-co-[AS—OH]_{z}),
poly(MMAₓ-co-[MAS—O—R—OH]_{y}-co-[MAS—OH]_{z}),
poly(EAₓ-co-[AS—O—R—OH]_{y}-co-[AS—OH]_{z}),
poly(EAₓ-co-[MAS—O—R—OH]_{y}-co-[MAS—OH]_{z}),
poly(EMAₓ-co-[AS—O—R—OH]_{y}-co-[AS—OH]_{z}) or
poly(EMAₓ-co-[MAS—O—R—OH]_{y}-co-[MAS—OH]_{z}).

3. A copolymer as claimed in claim 1, wherein it has the structure:
poly(MA₁-co-[MAS—O—R—OH]₁),
poly(MA₂-co-[MAS—O—R—OH]₁),
poly(MA₁-co-[MAS—O—R—OH]₂),
poly(MMA₁-co-[MAS—O—R—OH]₁),
poly(MMA₂-co-[MAS—O—R—OH]₁) or
poly(MMA₁-co-[MAS—O—R—OH]₂).

4. A copolymer as claimed in claim 1, wherein R=—CH₂(C═O)— or R=—CH(CH₃)(C═O)—.

5. A copolymer as claimed in claim 1, wherein it has a molar mass M_{w} with 4000 g·mol⁻¹≤M_{w}<500 000 g·mol⁻.

6. A copolymer as claimed in claim 1, wherein it has a polydispersity M̅_{w}/M̅ₙ≤3, M̅_{w}/M̅ₙ≤2.5, M̅_{w}/M̅ₙ≤2, M̅_{w}/M̅ₙ≤1.8 or M̅_{w}/M̅ₙ≤1.6.

7. The use of a copolymer as claimed in claim 1 in a pharmaceutical formulation or for coating of tablets or capsules.

8. A process for preparing a copolymer, comprising the steps of
(a) esterifying:
an α-hydroxycarboxylic acid selected from the group consisting of: hydroxyethanoic acid, 2-hydroxypropanoic acid, 2-hydroxybutanoic acid, 2-hydroxyisobutanoic acid, 2-hydroxy-2-methyl-3-oxobutanoic acid, phenylhydroxyethanoic acid, 2-hydroxy-4-methylthiobutanoic acid, 2-hydroxybutane-1,4-dioic acid, 2-hydroxypropanedioic acid, 2-hydroxypropane-1,2,3-tricarboxylic acid, hydroxypropane-1,2,3-tricarboxylic acid and 2,3-dihydroxybutanedioic acid having the structure
OH—R—OH
wherein
R=—CH₂(C═O)—, R=—CH(CH₃)(C═O)—, R=—CH(CH₂CH₃)(C═O)—, R=—C(CH₃)₂(C═O)—, R=—C(CH₃)(COCH₃)(C═O)—, R=—CH(Ph)(C═O)—, R=—CH[(CH₂)₂SCH₃](C═O)—, R=—CH(CH₂COOH)(C═O)—, R=—CH(COOH)(C═O)—, R=—C(CH₂COOH)₂(C═O)—, R=—CH(COOH)CH(CH₂COOH)(C═O)— or R=—CH(COOH)(CHOH)(C═O)—;
with
acrylic acid ((CH₂)HC—COOH) or methacrylic acid ((CH₂)(CH₃)C—COOH); to give a compound having the structure
Ayl-O—R—OH (Ia)
or
MAyl-O—R—OH (IIa)
wherein “Ayl”=acryloyl ((CH₂)HC—CO—) and “MAyl”=methacryloyl ((CH₂)(CH₃)C—CO—);
(b) optionally mono- or polyesterifying the compound (Ia) or (IIa) obtained in step (a) with an α-hydroxycarboxylic acid, in order to obtain a compound of the structure
Ayl-(O—R)ₘ—OH (Ib)
or
MAyl-(O—R)ₘ—OH (IIb)
wherein 2≤m≤20;
(c) conjugating the compound (Ia), (Ib), (IIa) or (IIb) obtained in step (a) or (b) with a protecting group P, in order to obtain a compound of the structure
Ayl-(O—R)ₙ—OP (Ic)
or
MAyl-(O—R)ₙ—OP (IIc)
with wherein 1≤n≤20;
(d) optionally conjugating acrylic acid or methacrylic acid with the protecting group P in order to obtain protected acrylic acid ((CH₂)HC—COOP) or protected methacrylic acid ((CH₂)(CH₃)C—COOP);
(e) polymerizing the compound (Ic) or (IIc) in a relative molar proportion y with an acrylate selected from the group consisting of: methyl acrylate, methyl methacrylate, ethyl acrylate and ethyl methacrylate,
in a relative molar proportion x and optionally with protected acrylic acid or protected methacrylic acid in a relative molar proportion z to give a copolymer of the following type:
poly(MAₓ-co-[AS—(O—R)ₙ—OP]_{y}-co-[AS—OP]_{z}),
poly(MMAₓ-co-[AS—(O—R)ₙ—OP]_{y}-co-[AS—OP]_{z}),
poly(EAₓ-co-[AS—(O—R)ₙ—OP]_{y}-co-[AS—OP]_{z}),
poly(EMAₓ-co-[AS—(O—R)ₙ—OP]_{y}-co-[AS—OP]_{z}),
poly(MAₓ-co-[AS—(O—R)ₙ—OP]_{y}-co-[MAS—OP]_{z}),
poly(MMAₓ-co-[AS—(O—R)ₙ—OP]_{y}-co-[MAS—OP]_{z}),
poly(EAₓ-co-[AS—(O—R)ₙ—OP]_{y}-co-[MAS—OP]_{z}),
poly(EMAₓ-co-[AS—(O—R)ₙ—OP]_{y}-co-[MAS—OP]_{z}),
poly(MAₓ-co-[MAS—(O—R)ₙ—OP]_{y}-co-[AS—OP]_{z}),
poly(MMAₓ-co-[MAS—(O—R)ₙ—OP]_{y}-co-[AS—OP]_{z}),
poly(EAₓ-co-[MAS—(O—R)ₙ—OP]_{y}-co[AS—OP]_{z}),
poly(EMAₓ-co-[MAS—(O—R)ₙ—OP]_{y}-co[AS—OP]_{z}),
poly(MAₓ-co-[MAS—(O—R)ₙ—OP]_{y}-co-[MAS—OP]_{z}),
poly(MMAₓ-co-[MAS—(O—R)ₙ—OP]_{y}-co[MAS—OP]_{z}),
poly(EAₓ-co-[MAS—(O—R)ₙ—OP]_{y}-co-[MAS—OP]_{z}),
or
poly(EMAₓ-co-[MAS—(O—R)ₙ—OP]_{y}-co-[MAS—OP]_{z}),
wherein MA=methyl acrylate residue (—CH[(C═O)OCH₃]CH₂—), MMA=methyl methacrylate residue (—C(CH₃)[(C═O)OCH₃]CH₂—), EA=ethyl acrylate residue (—CH[(C═O)OCH₂CH₃]CH₂—), EMA=ethyl methacrylate residue (—C(CH₃)[(C═O)OCH₂CH₃]CH₂—); AS=acrylic acid residue (—CH[(C═O)—]CH₂—), MAS=methacrylic acid residue (—C(CH₃)[(C═O)—]CH₂—); 1≤x≤20, 1≤y≤20 and 0≤z≤20;and
(f) deprotecting and hydrolyzing the copolymer obtained in step (e) in order to obtain a copolymer of the following type:
poly(MAₓ-co-[AS—(O—R)ₙ—OH]_{y}-co-[AS—OH]_{z}),
poly(MMAₓ-co-[AS—(O—R)ₙ—OH]_{y}-co-[AS—OH]_{z}),
poly(EAₓ-co-[AS—(O—R)ₙ—OH]_{y}-co-[AS—OH]_{z}),
poly(EMAₓ-co-[AS—(O—R)ₙ—OH]_{y}-co-[AS—OH]_{z}),
poly(MAₓ-co-[AS—(O—R)ₙ—OH]_{y}-co-[MAS—OH]_{z}),
poly(MMAₓ-co-[AS—(O—R)ₙ—OH]_{y}-co-[MAS—OH]_{z}),
poly(EAₓ-co-[AS—(O—R)ₙ—OH]_{y}-co-[MAS—OH]_{z}),
poly(EMAₓ-co-[AS—(O—R)ₙ—OH]_{y}-co-[MAS—OH]_{z}),
poly(MAₓ-co-[MAS—(O—R)ₙ—OH]_{y}-co-[AS—OH]_{z}),
poly(MMAₓ-co-[MAS—(O—R)ₙ—OH]_{y}-co-[AS—OH]_{z}),
poly(EAₓ-co-[MAS—(O—R)ₙ—OH]_{y}-co-[AS—OH]_{z}),
poly(EMAₓ-co-[MAS—(O—R)ₙ—OH]_{y}-co-[AS—OH]_{z})
poly(MAₓ-co-[MAS—(O—R)ₙ—OH]_{y}-co-[MAS—OH]_{z}),
poly(MMAₓ-co-[MAS—(O—R)ₙ—OH]_{y}-co-[MAS—OH]_{z}),
poly(EAₓ-co-[MAS—(O—R)ₙ—OH]_{y}-co-[MAS—OH]_{z}),
or
poly(EMAₓ-co-[MAS—(O—R)ₙ—OH]_{y}-co-[MAS—OH]_{z}).

9. The process as claimed in claim 8, wherein a free-radical polymerization is performed in step (e).

10. The process as claimed in claim 8, wherein a RAFT polymerization (reversible addition fragmentation chain transfer polymerization) using a chain transfer reagent is performed in step (e).

11. The process as claimed in claim 8, wherein the deprotection and hydrolysis in step (f) is performed using a catalyst.

12. A copolymer preparable by a process as claimed in claim 8.
